(19) United States
(12) Patent Application Publication
(10) Pub. No.: US 2026/0258141 A1
(43) Pub. Date: Sep. 3, 2026

(54) ANTI-IGF1R ANTIBODIES AND USES THEREOF

(71) Applicant: Biocytogen Pharmaceuticals (Beijing) Co., Ltd., Beijing (CN)

(72) Inventors: Wenqian An, Beijing (CN); Wenpang Cao, Beijing (CN); Rifei Li, Beijing (CN); Zilin Wang, Beijing (CN)

(21) Appl. No.: 19/163,474

(22) PCT Filed: Apr. 3, 2024
(86) PCT No.: PCT/CN2024/085734

§ 371 Date: Sep. 9, 2025

(30) Foreign Application Priority Data
Apr. 4, 2023 (WO) PCT/CN2023/086082
Aug. 28, 2023 (WO) PCT/CN2023/115246

(51) Int. Cl. C07K 16/28; A61K 38/00; A61K 45/06; A61K 47/68; A61P 35/00; C07K 14/705; C12N 5/071; C12N 15/63

(52) U.S. Cl. CPC C07K 16/2863; A61K 38/00; A61K 45/06; A61K 47/6803; A61P 35/00; C07K 14/70503; C12N 5/0682; C12N 15/63; C07K 2317/10; C07K 2317/24; C07K 2317/31; C07K 2317/565; C07K 2317/622; C07K 2317/732; C07K 2317/77; C07K 2317/94; C12N 2510/04

(57) ABSTRACT

Provided are anti-IGF1R (insulin-like growth factor 1 receptor) antibodies, antigen-binding fragments, and the uses thereof.

### CLAIM OF PRIORITY

This application claims priority to PCT/CN2023/086082, filed on Apr. 4, 2023, and PCT/CN2023/115246, filed on Aug. 28, 2023. The entire contents of the foregoing applications are incorporated herein by reference.

### TECHNICAL FIELD

This disclosure relates to anti-IGF1R (insulin-like growth factor 1 receptor) antibodies and uses thereof.

### BACKGROUND

Cancer is currently one of the diseases that have the highest human mortality. According to the World Health Organization statistical data, in 2012, the number of global cancer incidence and death cases reached 14 million and 8.2 million, respectively. In China, the newly diagnosed cancer cases are 3.07 million, and the death toll is 2.2 million. Recent clinical and commercial success of anticancer antibodies has created great interest in antibody-based therapeutics.

In addition, thyroid eye diseases (TEDs) such as thyroid-associated ophthalmopathy, a condition commonly associated with Graves' disease, remains inadequately treated. Current medical therapies, which primarily consist of glucocorticoids, have limited efficacy and present safety concerns.

There is a need to develop antibodies for use in various antibody-based therapeutics to treat cancers, auto-immune diseases, or eye diseases such as thyroid-associated ophthalmopathy.

### SUMMARY

This disclosure relates to anti-IGF1R antibodies, antigen-binding fragment thereof, and the uses thereof.

In one aspect, the disclosure is related to an antibody or antigen-binding fragment thereof that binds to insulin-like growth factor 1 receptor (IGF1R), comprising: a heavy chain variable region (VH) comprising complementarity determining regions (CDRs) 1, 2, and 3, in some embodiments, the VH CDR1 region comprises an amino acid sequence that is at least 80% identical to a selected VH CDR1 amino acid sequence, the VH CDR2 region comprises an amino acid sequence that is at least 80% identical to a selected VH CDR2 amino acid sequence, and the VH CDR3 region comprises an amino acid sequence that is at least 80% identical to a selected VH CDR3 amino acid sequence; and a light chain variable region (VL) comprising CDRs 1, 2, and 3, in some embodiments, the VL CDR1 region comprises an amino acid sequence that is at least 80% identical to a selected VL CDR1 amino acid sequence, the VL CDR2 region comprises an amino acid sequence that is at least 80% identical to a selected VL CDR2 amino acid sequence, and the VL CDR3 region comprises an amino acid sequence that is at least 80% identical to a selected VL CDR3 amino acid sequence, in some embodiments, the selected VH CDRs 1, 2, and 3 amino acid sequences and the selected VL CDRs, 1, 2, and 3 amino acid sequences are one of the following:
(1) the selected VH CDRs 1, 2, 3 amino acid sequences are set forth in SEQ ID NOs: 1, 2, 3, respectively, and the selected VL CDRs 1, 2, 3 amino acid sequences are set forth in SEQ ID NOs: 4, 5, 6, respectively;
(2) the selected VH CDRs 1, 2, 3 amino acid sequences are set forth in SEQ ID NOs: 25, 26, 27, respectively, and the selected VL CDRs 1, 2, 3 amino acid sequences are set forth in SEQ ID NOs: 4, 5, 6, respectively;
(3) the selected VH CDRs 1, 2, 3 amino acid sequences are set forth in SEQ ID NOs: 7, 8, 9, respectively, and the selected VL CDRs 1, 2, 3 amino acid sequences are set forth in SEQ ID NOs: 10, 11, 12, respectively;
(4) the selected VH CDRs 1, 2, 3 amino acid sequences are set forth in SEQ ID NOs: 28, 29, 30, respectively, and the selected VL CDRs 1, 2, 3 amino acid sequences are set forth in SEQ ID NOs: 10, 11, 12, respectively;
(5) the selected VH CDRs 1, 2, 3 amino acid sequences are set forth in SEQ ID NOs: 13, 14, 15, respectively, and the selected VL CDRs 1, 2, 3 amino acid sequences are set forth in SEQ ID NOs: 16, 17, 18, respectively;
(6) the selected VH CDRs 1, 2, 3 amino acid sequences are set forth in SEQ ID NOs: 31, 32, 33, respectively, and the selected VL CDRs 1, 2, 3 amino acid sequences are set forth in SEQ ID NOs: 16, 17, 18, respectively;
(7) the selected VH CDRs 1, 2, 3 amino acid sequences are set forth in SEQ ID NOs: 19, 20, 21, respectively, and the selected VL CDRs 1, 2, 3 amino acid sequences are set forth in SEQ ID NOs: 22, 23, 24, respectively;
(8) the selected VH CDRs 1, 2, 3 amino acid sequences are set forth in SEQ ID NOs: 34, 35, 36, respectively, and the selected VL CDRs 1, 2, 3 amino acid sequences are set forth in SEQ ID NOs: 22, 23, 24, respectively.

In some embodiments, the VH comprises CDRs 1, 2, 3 with the amino acid sequences set forth in SEQ ID NOs: 1, 2, and 3, respectively, and the VL comprises CDRs 1, 2, 3 with the amino acid sequences set forth in SEQ ID NOs: 4, 5, and 6, respectively, according to Kabat definition. In some embodiments, the VH comprises CDRs 1, 2, 3 with the amino acid sequences set forth in SEQ ID NOs: 7, 8, and 9, respectively, and the VL comprises CDRs 1, 2, 3 with the amino acid sequences set forth in SEQ ID NOs: 10, 11, and 12, respectively, according to Kabat definition. In some embodiments, the VH comprises CDRs 1, 2, 3 with the amino acid sequences set forth in SEQ ID NOs: 13, 14, and 15, respectively, and the VL comprises CDRs 1, 2, 3 with the amino acid sequences set forth in SEQ ID NOs: 16, 17 and 18, respectively, according to Kabat definition. In some embodiments, the VH comprises CDRs 1, 2, 3 with the amino acid sequences set forth in SEQ ID NOs: 19, 20, and 21, respectively, and the VL comprises CDRs 1, 2, 3 with the amino acid sequences set forth in SEQ ID NOs: 22, 23, and 24, respectively, according to Kabat definition. In some embodiments, the VH comprises CDRs 1, 2, 3 with the amino acid sequences set forth in SEQ ID NOs: 25, 26, and 27, respectively, and the VL comprises CDRs 1, 2, 3 with the amino acid sequences set forth in SEQ ID NOs: 4, 5, and 6, respectively, according to Chothia definition. In some embodiments, the VH comprises CDRs 1, 2, 3 with the amino acid sequences set forth in SEQ ID NOs: 28, 29, and 30, respectively, and the VL comprises CDRs 1, 2, 3 with the amino acid sequences set forth in SEQ ID NOs: 10, 11, and 12, respectively, according to Chothia definition. In some embodiments, the VH comprises CDRs 1, 2, 3 with the amino acid sequences set forth in SEQ ID NOs: 31, 32, and 33, respectively, and the VL comprises CDRs 1, 2, 3 with the amino acid sequences set forth in SEQ ID NOs: 16, 17 and 18, respectively, according to Chothia definition. In some embodiments, the VH comprises CDRs 1, 2, 3 with the amino acid sequences set forth in SEQ ID NOs: 34, 35, and 36, respectively, and the VL comprises CDRs 1, 2, 3 with the amino acid sequences set forth in SEQ ID NOs: 22, 23, and 24, respectively, according to Chothia definition.

In some embodiments, the antibody or antigen-binding fragment specifically binds to human IGF1R or monkey IGF1R. In some embodiments, the antibody or antigen-binding fragment is a human or humanized antibody or antigen-binding fragment thereof (e.g., a human IgG1 or IgG4 antibody). In some embodiments, the antibody or antigen-binding fragment is a single-chain variable fragment (scFv) or a multi-specific antibody (e.g., a bispecific antibody).

In one aspect, the disclosure is related to a nucleic acid comprising a polynucleotide encoding a polypeptide comprising:
(1) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 1, 2, and 3, respectively, and wherein the VH, when paired with a light chain variable region (VL) comprising the amino acid sequence set forth in SEQ ID NO: 38 binds to IGF1R;
(2) an immunoglobulin light chain or a fragment thereof comprising a VL comprising CDRs 1, 2, and 3 comprising the amino acid sequences set forth in SEQ ID NOs: 4, 5, and 6, respectively, and wherein the VL, when paired with a VH comprising the amino acid sequence set forth in SEQ ID NO: 37 binds to IGF1R;
(3) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 25, 26, and 27, respectively, and wherein the VH, when paired with a light chain variable region (VL) comprising the amino acid sequence set forth in SEQ ID NO: 38 binds to IGF1R;
(4) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 7, 8, and 9, respectively, and wherein the VH, when paired with a light chain variable region (VL) comprising the amino acid sequence set forth in SEQ ID NO: 40 binds to IGF1R;
(5) an immunoglobulin light chain or a fragment thereof comprising a VL comprising CDRs 1, 2, and 3 comprising the amino acid sequences set forth in SEQ ID NOs: 10, 11, and 12, respectively, and wherein the VL, when paired with a VH comprising the amino acid sequence set forth in SEQ ID NO: 39 binds to IGF1R;
(6) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 28, 29, and 30, respectively, and wherein the VH, when paired with a light chain variable region (VL) comprising the amino acid sequence set forth in SEQ ID NO: 40 binds to IGF1R;
(7) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 13, 14, and 15, respectively, and wherein the VH, when paired with a light chain variable region (VL) comprising the amino acid sequence set forth in SEQ ID NO: 42 binds to IGF1R;
(8) an immunoglobulin light chain or a fragment thereof comprising a VL comprising CDRs 1, 2, and 3 comprising the amino acid sequences set forth in SEQ ID NOs: 16, 17, and 18, respectively, and wherein the VL, when paired with a VH comprising the amino acid sequence set forth in SEQ ID NO: 41 binds to IGF1R;
(9) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 31, 32, and 33, respectively, and wherein the VH, when paired with a light chain variable region (VL) comprising the amino acid sequence set forth in SEQ ID NO: 42 binds to IGF1R;
(10) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 19, 20 and 21, respectively, and wherein the VH, when paired with a light chain variable region (VL) comprising the amino acid sequence set forth in SEQ ID NO: 44 binds to IGF1R;
(11) an immunoglobulin light chain or a fragment thereof comprising a VL comprising CDRs 1, 2, and 3 comprising the amino acid sequences set forth in SEQ ID NOs: 22, 23, and 24, respectively, and wherein the VL, when paired with a VH comprising the amino acid sequence set forth in SEQ ID NO: 43 binds to IGF1R; or
(12) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 34, 35, and 36, respectively, and wherein the VH, when paired with a light chain variable region (VL) comprising the amino acid sequence set forth in SEQ ID NO: 44 binds to IGF1R.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 1, 2, and 3, respectively, or SEQ ID NOs: 25, 26, and 27, respectively. In some embodiments, the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 4, 5, and 6, respectively.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 7, 8, and 9, respectively, or SEQ ID NOs: 28, 29, and 30, respectively. In some embodiments, the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 10, 11, and 12, respectively.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 13, 14, and 15, respectively, or SEQ ID NOs: 31, 32, and 33, respectively. In some embodiments, the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 16, 17, and 18, respectively.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 19, 20, and 21, respectively, or SEQ ID NOs: 34, 35, and 36, respectively. In some embodiments, the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 22, 23, and 24, respectively.

In some embodiments, the VH when paired with a VL specifically binds to human or monkey IGF1R, or the VL when paired with a VH specifically binds to human IGF1R or monkey IGF1R. In some embodiments, the immunoglobulin heavy chain or the fragment thereof is a human or humanized immunoglobulin heavy chain or a fragment thereof (e.g., a human IgG1 heavy chain or a fragment thereof), and the immunoglobulin light chain or the fragment thereof is a human or humanized immunoglobulin light chain or a fragment thereof. In some embodiments, the nucleic acid encodes a single-chain variable fragment (scFv), a multi-specific antibody (e.g., a bispecific antibody) or a chimeric antigen receptor (CAR). In some embodiments, the nucleic acid is cDNA.

In one aspect, the disclosure is related to a vector comprising one or more of the nucleic acids described herein. In one aspect, the disclosure is related to a vector comprising two of the nucleic acids described herein, in some embodiments, the vector encodes the VH region and the VL region that together bind to IGF1R. In one aspect, the disclosure is related to a pair of vectors, in some embodiments, each vector comprises one of the nucleic acids described herein, in some embodiments, together the pair of vectors encodes the VH region and the VL region that together bind to IGF1R.

In one aspect, the disclosure is related to a cell comprising the vector or the pair of vectors described herein. In some embodiments, the cell is a CHO cell. In one aspect, the disclosure is related to a cell comprising one or more of the nucleic acids described herein. In one aspect, the disclosure is related to a cell comprising two of the nucleic acids described herein. In some embodiments, the two nucleic acids together encode the VH region and the VL region that together bind to IGF1R.

In one aspect, the disclosure is related to a method of producing an antibody or an antigen-binding fragment thereof, the method comprising (a) culturing the cell described herein under conditions sufficient for the cell to produce the antibody or the antigen-binding fragment; and (b) collecting the antibody or the antigen-binding fragment produced by the cell.

In one aspect, the disclosure is related to an antibody or antigen-binding fragment thereof that binds to IGF1R comprising a heavy chain variable region (VH) comprising an amino acid sequence that is at least 80% identical to a selected VH sequence, and a light chain variable region (VL) comprising an amino acid sequence that is at least 80% identical to a selected VL sequence, in some embodiments, the selected VH sequence and the selected VL sequence are one of the following: (1) the selected VH sequence is SEQ ID NO: 37, and the selected VL sequence is SEQ ID NO: 38; (2) the selected VH sequence is SEQ ID NO: 39, and the selected VL sequence is SEQ ID NO: 40; (3) the selected VH sequence is SEQ ID NO: 41, and the selected VL sequence is SEQ ID NO: 42; and (4) the selected VH sequence is SEQ ID NO: 43, and the selected VL sequence is SEQ ID NO: 44.

In some embodiments, the VH comprises the sequence of SEQ ID NO: 37 and the VL comprises the sequence of SEQ ID NO: 38. In some embodiments, the VH comprises the sequence of SEQ ID NO: 39 and the VL comprises the sequence of SEQ ID NO: 40. In some embodiments, the VH comprises the sequence of SEQ ID NO: 41 and the VL comprises the sequence of SEQ ID NO: 42. In some embodiments, the VH comprises the sequence of SEQ ID NO: 43 and the VL comprises the sequence of SEQ ID NO: 44.

In some embodiments, the antibody or antigen-binding fragment specifically binds to human IGF1R or monkey IGF1R. In some embodiments, the antibody or antigen-binding fragment is a human or humanized antibody or antigen-binding fragment thereof. In some embodiments, the antibody or antigen-binding fragment is a single-chain variable fragment (scFv) or a multi-specific antibody (e.g., a bispecific antibody).

In one aspect, the disclosure is related to an antibody or antigen-binding fragment thereof that cross-competes with the antibody or antigen-binding fragment thereof described herein.

In one aspect, the disclosure is related to an antibody or antigen-binding fragment thereof that binds to IGF1R comprising a heavy chain variable region (VH) comprising VH CDR1, VH CDR2, and VH CDR3 that are identical to VH CDR1, VH CDR2, and VH CDR3 of a selected VH sequence; and a light chain variable region (VL) comprising VL CDR1, VL CDR2, and VL CDR3 that are identical to VL CDR1, VL CDR2, and VL CDR3 of a selected VL sequence, in some embodiments, the selected VH sequence and the selected VL sequence are one of the following: (1) the selected VH sequence is SEQ ID NO: 37, and the selected VL sequence is SEQ ID NO: 38; (2) the selected VH sequence is SEQ ID NO: 39, and the selected VL sequence is SEQ ID NO: 40; (3) the selected VH sequence is SEQ ID NO: 41, and the selected VL sequence is SEQ ID NO: 42; and (4) the selected VH sequence is SEQ ID NO: 43, and the selected VL sequence is SEQ ID NO: 44.

In one aspect, the disclosure is related to an antibody-drug conjugate comprising the antibody or antigen-binding fragment thereof described herein covalently bound to a therapeutic agent. In some embodiments, the therapeutic agent is a cytotoxic or cytostatic agent.

In one aspect, the disclosure is related to a method of treating a subject having cancer, the method comprising administering a therapeutically effective amount of a composition comprising the antibody or antigen-binding fragment thereof or the antibody-drug conjugate described herein, to the subject. In some embodiments, the subject has a solid tumor, brain cancer, lung cancer, melanoma, prostate cancer, esophageal squamous cell carcinoma, leukemia, lymphoma, ovarian cancer, colorectal cancer, head and neck cancer, bladder cancer, renal cancer and breast cancer. In some embodiments, the method described herein further comprises administering a therapeutically effective amount of an anti-OX40 antibody, an anti-PD1 antibody, an anti-PDL1 antibody, an anti-PDL2 antibody, an anti-LAG-3 antibody, an anti-TIGIT antibody, an anti-CTLA-4 antibody, an anti-GITR antibody, an anti-TIM-3 antibody, an anti-4-1BB antibody, and/or an anti-CD40 antibody, to the subject.

In one aspect, the disclosure is related to a method of decreasing the rate of tumor growth, the method comprising contacting a tumor cell with an effective amount of a composition comprising an antibody or antigen-binding fragment thereof or the antibody-drug conjugate described herein.

In one aspect, the disclosure is related to a method of killing a tumor cell, the method comprising contacting a tumor cell with an effective amount of a composition comprising the antibody or antigen-binding fragment thereof or the antibody-drug conjugate described herein.

In one aspect, the disclosure is related to a method of treating a subject having an eye disease or disorder, the method comprising administering a therapeutically effective amount of a composition comprising the antibody or antigen-binding fragment thereof or the antibody-drug conjugate described herein, to the subject.

In some embodiments, the subject has thyroid eye disease (TED). In some embodiments, the thyroid eye disease is thyroid-associated ophthalmopathy.

In one aspect, the disclosure is related to a method of treating a subject having a bone disease or disorder, the method comprising administering a therapeutically effective amount of a composition comprising the antibody or antigen-binding fragment thereof or the antibody-drug conjugate described herein, to the subject.

In one aspect, the disclosure is related to a pharmaceutical composition comprising the antibody or antigen-binding fragment thereof described herein and a pharmaceutically acceptable carrier. In one aspect, the disclosure is related to a pharmaceutical composition comprising the antibody drug conjugate described herein, and a pharmaceutically acceptable carrier.

In some embodiments, the concentration of the antibody is about 50 to about 150 mg/ml (e.g., about 100 mg/ml).

In one aspect, the disclosure is related to a chimeric antigen receptor (CAR) comprising the antibody or antigen-binding fragment thereof described herein.

As used herein, a “thyroid eye disease (TED)” is a condition in which the eye muscles, eyelids, tear glands and fatty tissues behind the eye become inflamed. The symptoms of a thyroid eye disease include, but are not limited to redness of eyes and eyelids, swollen, “staring” or “bulging” eyes.

As used herein, the term “antibody” refers to any antigen-binding molecule that contains at least one (e.g., one, two, three, four, five, or six) complementary determining region (CDR) (e.g., any of the three CDRs from an immunoglobulin light chain or any of the three CDRs from an immunoglobulin heavy chain) and is capable of specifically binding to an epitope. Non-limiting examples of antibodies include: monoclonal antibodies, polyclonal antibodies, multi-specific antibodies (e.g., bi-specific antibodies), single-chain antibodies, chimeric antibodies, human antibodies, and humanized antibodies. In some embodiments, an antibody can contain an Fc region of a human antibody. The term antibody also includes derivatives, e.g., bi-specific antibodies, single-chain antibodies, diabodies, linear antibodies, and multi-specific antibodies formed from antibody fragments.

As used herein, the term “antigen-binding fragment” refers to a portion of a full-length antibody, wherein the portion of the antibody is capable of specifically binding to an antigen. In some embodiments, the antigen-binding fragment contains at least one variable domain (e.g., a variable domain of a heavy chain or a variable domain of light chain). Non-limiting examples of antibody fragments include, e.g., Fab, Fab′, F(ab′)₂, and Fv fragments.

As used herein, the term “human antibody” refers to an antibody that is encoded by an endogenous nucleic acid (e.g., rearranged human immunoglobulin heavy or light chain locus) derived from a human. In some embodiments, a human antibody is collected from a human or produced in a human cell culture (e.g., human hybridoma cells). In some embodiments, a human antibody is produced in a non-human cell (e.g., a mouse or hamster cell line). In some embodiments, a human antibody is produced in a bacterial or yeast cell. In some embodiments, a human antibody is produced in a transgenic non-human animal (e.g., a bovine) containing an unrearranged or rearranged human immunoglobulin locus (e.g., heavy or light chain human immunoglobulin locus).

As used herein, the term “chimeric antibody” refers to an antibody that contains a sequence present in at least two different species (e.g., antibodies from two different mammalian species such as a human and a mouse antibody). A non-limiting example of a chimeric antibody is an antibody containing the variable domain sequences (e.g., all or part of a light chain and/or heavy chain variable domain sequence) of a non-human (e.g., mouse) antibody and the constant domains of a human antibody. Additional examples of chimeric antibodies are described herein and are known in the art.

As used herein, the term “humanized antibody” refers to a non-human antibody which contains minimal sequence derived from a non-human (e.g., mouse) immunoglobulin and contains sequences derived from a human immunoglobulin. In non-limiting examples, humanized antibodies are human antibodies (recipient antibody) in which hypervariable (e.g., CDR) region residues of the recipient antibody are replaced by hypervariable (e.g., CDR) region residues from a non-human antibody (e.g., a donor antibody), e.g., a mouse, rat, or rabbit antibody, having the desired specificity, affinity, and capacity. In some embodiments, the Fv framework residues of the human immunoglobulin are replaced by corresponding non-human (e.g., mouse) immunoglobulin residues. In some embodiments, humanized antibodies may contain residues which are not found in the recipient antibody or in the donor antibody. These modifications can be made to further refine antibody performance. In some embodiments, the humanized antibody contains substantially all of at least one, and typically two, variable domains, in which all or substantially all of the hypervariable loops (CDRs) correspond to those of a non-human (e.g., mouse) immunoglobulin and all or substantially all of the framework regions are those of a human immunoglobulin. The humanized antibody can also contain at least a portion of an immunoglobulin constant region (Fc), typically, that of a human immunoglobulin. Humanized antibodies can be produced using molecular biology methods known in the art. Non-limiting examples of methods for generating humanized antibodies are described herein.

As used herein, the term “single-chain antibody” refers to a single polypeptide that contains at least two immunoglobulin variable domains (e.g., a variable domain of a mammalian immunoglobulin heavy chain or light chain) that is capable of specifically binding to an antigen. Non-limiting examples of single-chain antibodies are described herein.

As used herein, the terms “subject” and “patient” are used interchangeably throughout the specification and describe an animal, human or non-human, to whom treatment according to the methods of the present invention is provided. Veterinary and non-veterinary applications are contemplated in the present disclosure. Human patients can be adult humans or juvenile humans (e.g., humans below the age of 18 years old). In addition to humans, patients include but are not limited to mice, rats, hamsters, guinea-pigs, rabbits, ferrets, cats, dogs, and primates. Included are, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

As used herein, when referring to an antibody, the phrases “specifically binding” and “specifically binds” mean that the antibody interacts with its target molecule (e.g., IGF1R) preferably to other molecules, because the interaction is dependent upon the presence of a particular structure (i.e., the antigenic determinant or epitope) on the target molecule; in other words, the reagent is recognizing and binding to molecules that include a specific structure rather than to all molecules in general. An antibody that specifically binds to the target molecule may be referred to as a target-specific antibody. For example, an antibody that specifically binds to a IGF1R molecule may be referred to as a IGF1R-specific antibody or an anti-IGF1R antibody.

As used herein, the terms “polypeptide,” “peptide,” and “protein” are used interchangeably to refer to polymers of amino acids of any length of at least two amino acids.

As used herein, the terms “polynucleotide,” “nucleic acid molecule,” and “nucleic acid sequence” are used interchangeably herein to refer to polymers of nucleotides of any length of at least two nucleotides, and include, without limitation, DNA, RNA, DNA/RNA hybrids, and modifications thereof.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 lists Kabat CDR sequences for anti-IGF1R antibodies.

FIG. 2 lists Chothia CDR sequences for anti-IGF1R antibodies.

FIG. 3 lists sequences of the heavy chain variable regions and light chain variable regions of the anti-IGF1R antibodies and amino acids sequences discussed in the disclosure.

FIG. 4 shows the internalization of anti-IGF1R antibodies (12A4, 12D1, 10D1, and 10G1). Teprotumumab analog was used as controls.

FIGS. 5A-5B show the inhibition of IGF1R autophosphorylation by the anti-IGF1R antibodies (12A4, 12D1, 10D1, and 10G1). Teprotumumab analog was used as controls.

FIG. 6 shows the inhibition of IGF-1-induced OPN secretion by the anti-IGF1R antibodies (12A4, 12D1, 10D1, and 10G1). Teprotumumab analog was used as a control.

FIG. 7 shows the pharmacokinetic characteristics of the anti-IGF1R antibodies.

FIG. 8 shows the ADCC cytotoxicity of anti-IGF1R antibodies (12A4-YTE and 12D1-QL). Teprotumumab analog was used as a positive control.

FIG. 9 shows the cytokine INF-y release levels treated with anti-IGF1R antibodies (12A4-YTE and 12D1-QL). Teprotumumab analog was used as a control.

FIG. 10 shows the HA (hyaluronan) release levels treated with anti-IGF1R antibodies (12A4-YTE and 12D1-QL). Teprotumumab analog was used as a control.

FIG. 11 shows the HA (hyaluronan) release levels treated with anti-IGF1R antibody 12A4-YTE. Teprotumumab analog was used as a control.

### DETAILED DESCRIPTION

The insulin-like growth factor 1 receptor (IGF1R) is a ubiquitously expressed membrane-bound tyrosine kinase receptor that recognizes its two major ligands IGF1 and IGF2, and controls multiple essential cellular functions. IGF activity is highly relevant in several chronic lung pathologies, including lung cancer. IGF1R signaling has been profusely implicated as a critical contributor to cancer cell proliferation, survival, migration, and resistance to anticancer therapies, thus targeting IGF signaling is an attractive therapeutic strategy. IGF1R has also been shown to be associated with thyroid eye diseases (TEDs) and bone diseases such as osteoporosis and osteosarcoma. Thus, anti-IGF1R antibodies can be potentially used as cancer therapies and treatment of other diseases such as eye diseases or bone diseases.

The present disclosure provides examples of antibodies, antigen-binding fragment thereof, that bind to IGF1R (insulin-like growth factor 1 receptor).

### IGF1R

IGF1R (insulin-like growth factor 1 receptor; UniProt Protein database for Accession No. P08069, IGF1R_HUMAN) is a receptor tyrosine kinase which mediates actions of insulin-like growth factor 1 (IGF-1). It binds IGF-1 with high affinity and IGF-2 and insulin (INS) with a lower affinity. The activated IGF1R is involved in cell growth and survival control. IGF1R is crucial for tumor transformation and survival of malignant cell. Ligand binding activates the receptor kinase, leading to receptor autophosphorylation, and tyrosines phosphorylation of multiple substrates, that function as signaling adapter proteins including, the insulin-receptor substrates (IRS1/2), Shc and 14-3-3 proteins. Phosphorylation of IRSs proteins lead to the activation of two main signaling pathways: the PI3K-AKT/PKB pathway and the Ras-MAPK pathway. The result of activating the MAPK pathway is increased cellular proliferation, whereas activating the PI3K pathway inhibits apoptosis and stimulates protein synthesis. Phosphorylated IRS1 can activate the 85 kDa regulatory subunit of PI3K (PIK3R1), leading to activation of several downstream substrates, including protein AKT/PKB. AKT phosphorylation, in turn, enhances protein synthesis through mTOR activation and triggers the antiapoptotic effects of IGF1R through phosphorylation and inactivation of BAD. In parallel to PI3K-driven signaling, recruitment of Grb2/SOS by phosphorylated IRS1 or She leads to recruitment of Ras and activation of the ras-MAPK pathway. In addition to these two main signaling pathways IGF1R signals also through the Janus kinase/signal transducer and activator of transcription pathway (JAK/STAT). Phosphorylation of JAK proteins can lead to phosphorylation/activation of signal transducers and activators of transcription (STAT) proteins. In particular activation of STAT3, may be essential for the transforming activity of IGF1R. The JAK/STAT pathway activates gene transcription and may be responsible for the transforming activity. JNK kinases can also be activated by the IGF1R. IGF1 exerts inhibiting activities on JNK activation via phosphorylation and inhibition of MAP3K5/ASK1, which is able to directly associate with the IGF1R.

IGF1R is currently being evaluated as a pharmacological target in clinical trials for oncologic patients, including non-small cell lung cancer (NSCLC). The role IGF1R plays in implantation and progression of lung metastases in the TME has been studied using the Lewis lung carcinoma (LLC) model as a reproducible syngeneic model for NSCLC. The implication of IGF1R as a cancer-promoting factor in the TME has been studied by analyzing tumor samples from NSCLC patients, and generating LLC models by performing heterotopic transplantation or pulmonary metastasis in the context of IGF1R deficiency.

Thyroid-associated ophthalmopathy, a condition commonly associated with Graves' disease, remains inadequately treated. Current medical therapies, which primarily consist of glucocorticoids, have limited efficacy and present safety concerns. Inhibition of the insulin-like growth factor I receptor (IGF-IR) can be a new therapeutic strategy to attenuate the underlying autoimmune pathogenesis of ophthalmopathy.

IGF1R was also found to synergistically interact with TSHR to increase osteopontin (OPN) secretion in bone diseases. Thyroid-stimulating hormone (TSH), also known as thyrotropin, is a hormone that activates TSH receptors (TSHRs) to stimulate development of the thyroid in utero and on thyroid cells to stimulate production of thyroid hormones thyroxine (T4) and triiodothyronine (T3) in the adult. Thyroid hormones are essential for skeletal development and healthy bone metabolism. Clinical studies have demonstrated that euthyroid status in the adult is important for bone homeostasis. Hyperthyroidism leads to increased bone resorption which causes reduced bone and its mineralization.

More information related to IGF1R can be found e.g., in Boutin et al., Front. Endocrinol., 20 May 2020 Sec. Thyroid Endocrinology; Girnita et al., Cell Mol Life Sci. 2014; 71:2403-27; Wang et al., Biomed Res Int. 2018; 2018:6057589; and Griffiths et al., Nature. 2020; 583:615-9; Ullrich et al., EMBO J. 5:2503-2512 (1986); and Kato et al., J. Biol. Chem. 268:2655-2661 (1993); Kellar et al., Biomed Res Int. 2015; 2015:621324; Alfaro-Arnedo et al., Oncogene volume 41, pages 3625-3639, 2022; Smith et al., N Engl J Med. 2017 May 4; 376(18):1748-1761; and Barbesino et al., The Journal of Clinical Endocrinology & Metabolism, Volume 107, Issue Supplement_1, September 2022, Pages S47-S56; each of which is incorporated herein by reference in the entirety.

### Anti-IGF1R Antibodies and Antigen-Binding Fragments

The disclosure provides antibodies and antigen-binding fragments thereof that specifically bind to IGF1R (e.g., human IGF1R). The antibodies and antigen-binding fragments described herein are capable of binding to IGF1R. In some embodiments, these antibodies block the binding of human IGF1R to IGF1R ligand (e.g., human insulin-like growth factor 1, IGF-1 or human insulin-like growth factor 2, IGF-2). In some embodiments, these antibodies inhibit human IGF-1-induced cell proliferation. In some embodiments, these antibodies block the IGF-1/IGF1R or IGF-2/IGF1R signaling pathway. In some embodiments, these antibodies can initiate complement-dependent cytotoxicity (CDC) or antibody-dependent cellular cytotoxicity (ADCC). In some embodiments, these antibodies bind to cells expressing IGF1R.

The disclosure provides, e.g., anti-IGF1R antibodies 12A4, 12D1, 10D1, 10G1, the modified antibodies thereof, including, e.g., chimeric antibodies, humanized antibodies, and human antibodies.

The CDR sequences for 12A4, and 12A4 derived antibodies (e.g., humanized antibodies) include CDRs of the heavy chain variable domain, SEQ ID NOs: 1, 2, 3, and CDRs of the light chain variable domain, SEQ ID NOs: 4, 5, 6, as defined by Kabat definition. The CDRs can also be defined by Chothia system. Under the Chothia definition, the CDR sequences of the heavy chain variable domain are set forth in SEQ ID NOs: 25, 26, 27, and CDR sequences of the light chain variable domain are set forth in SEQ ID NOs: 4, 5, 6.

The CDR sequences for 12D1, and 12D1 derived antibodies include CDRs of the heavy chain variable domain, SEQ ID NOs: 7, 8, 9, and CDRs of the light chain variable domain, SEQ ID NOs: 10, 11, 12, as defined by Kabat definition. Under Chothia definition, the CDR sequences of the heavy chain variable domain are set forth in SEQ ID NOs: 28, 29, 30, and CDRs of the light chain variable domain are set forth in SEQ ID NOs: 10, 11, 12.

The CDR sequences for 10D1, and 10D1 derived antibodies include CDRs of the heavy chain variable domain, SEQ ID NOs: 13, 14, 15, and CDRs of the light chain variable domain, SEQ ID NOs: 16, 17, 18, as defined by Kabat definition. Under Chothia definition, the CDR sequences of the heavy chain variable domain are set forth in SEQ ID NOs: 31, 32, 33, and CDRs of the light chain variable domain are set forth in SEQ ID NOs: 16, 17, 18.

The CDR sequences for 10G1, and 10G1 derived antibodies include CDRs of the heavy chain variable domain, SEQ ID NOs: 19, 20, 21, and CDRs of the light chain variable domain, SEQ ID NOs: 22, 23, 24, as defined by Kabat definition. Under Chothia definition, the CDR sequences of the heavy chain variable domain are set forth in SEQ ID NOs: 34, 35, 36, and CDRs of the light chain variable domain are set forth in SEQ ID NOs: 22, 23, 24.

The amino acid sequence for the heavy chain variable region of 12A4 antibody is set forth in SEQ ID NO: 37. The amino acid sequence for the light chain variable region of 12A4 antibody is set forth in SEQ ID NO: 38.

The amino acid sequence for the heavy chain variable region of 12D1 antibody is set forth in SEQ ID NO: 39. The amino acid sequence for the light chain variable region of 12D1 antibody is set forth in SEQ ID NO: 40.

The amino acid sequence for the heavy chain variable region of 10D1 antibody is set forth in SEQ ID NO: 41. The amino acid sequence for the light chain variable region of 10D1 antibody is set forth in SEQ ID NO: 42.

The amino acid sequence for the heavy chain variable region of 10G1 antibody is set forth in SEQ ID NO: 43. The amino acid sequence for the light chain variable region of 10G1 antibody is set forth in SEQ ID NO: 44.

The amino acid sequences for heavy chain variable regions and light variable regions of the modified antibodies are also provided. In some embodiments, the heavy chain variable region is at least 80%, 85%, 90%, 91%, 92%, 93%, 94%, 95%, 96%, 97%, 98%, or 99% identical to SEQ ID NO: 37, 39, 41 or 43. In some embodiments, the light chain variable region is at least 80%, 85%, 90%, 91%, 92%, 93%, 94%, 95%, 96%, 97%, 98%, or 99% identical to SEQ ID NO: 38, 40, 42 or 44. The heavy chain variable region sequence can be paired with the corresponding light chain variable region sequence, and together they bind to IGF1R.

In some embodiments, the variable regions are fully human, e.g., derived from human heavy chain immunoglobulin locus sequences (e.g., recombination of human IGHV, IGHD, and IGHJ genes), and/or human kappa chain immunoglobulin locus sequences (e.g., recombination of human IGKV and IGKJ genes).

Furthermore, in some embodiments, the antibodies or antigen-binding fragments thereof described herein can also contain one, two, or three heavy chain variable region CDRs selected from the group of SEQ ID NOs: 1-3, SEQ ID NOs: 7-9, SEQ ID NOs: 13-15, SEQ ID NOs: 19-21, SEQ ID NOS: 25-27, SEQ ID NOs: 28-30, SEQ ID NOs: 31-33, SEQ ID NOs: 34-36; and/or one, two, or three light chain variable region CDRs selected from the group of SEQ ID NOs: 4-6, SEQ ID NOs: 10-12, SEQ ID NOs: 16-18 and SEQ ID NOs: 22-24.

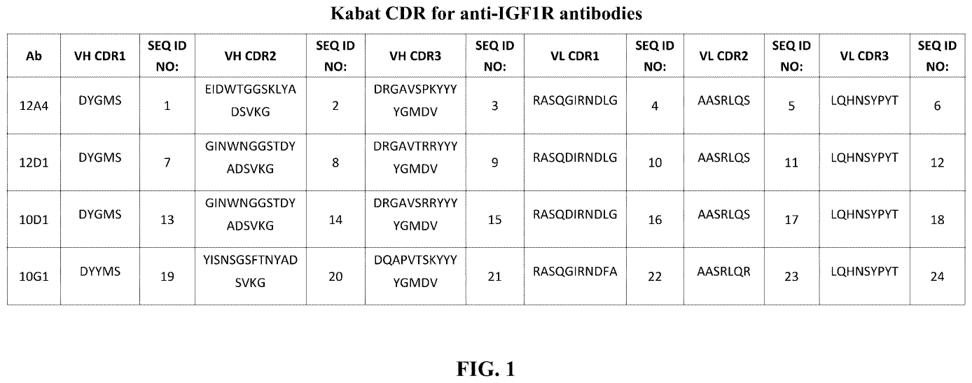

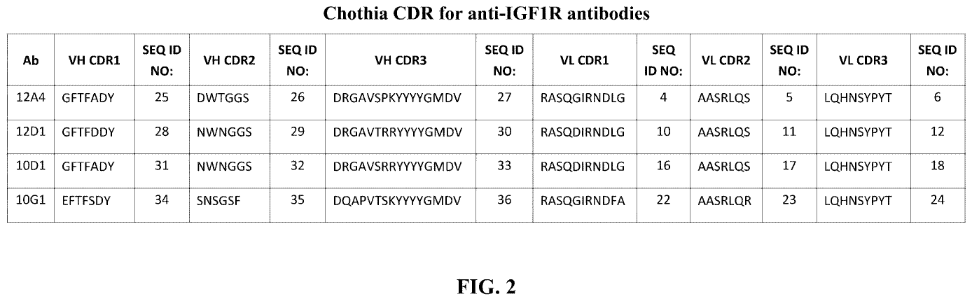

In some embodiments, the antibodies can have a heavy chain variable region (VH) comprising complementarity determining regions (CDRs) 1, 2, 3, wherein the CDR1 region comprises or consists of an amino acid sequence that is at least 80%, 85%, 90%, or 95% identical to a selected VH CDR1 amino acid sequence, the CDR2 region comprises or consists of an amino acid sequence that is at least 80%, 85%, 90%, or 95% identical to a selected VH CDR2 amino acid sequence, and the CDR3 region comprises or consists of an amino acid sequence that is at least 80%, 85%, 90%, or 95% identical to a selected VH CDR3 amino acid sequence. In some embodiments, the antibodies can have a light chain variable region (VL) comprising CDRs 1, 2, 3, wherein the CDR1 region comprises or consists of an amino acid sequence that is at least 80%, 85%, 90%, or 95% identical to a selected VL CDR1 amino acid sequence, the CDR2 region comprises or consists of an amino acid sequence that is at least 80%, 85%, 90%, or 95% identical to a selected VL CDR2 amino acid sequence, and the CDR3 region comprises or consists of an amino acid sequence that is at least 80%, 85%, 90%, or 95% identical to a selected VL CDR3 amino acid sequence. The selected VH CDRs 1, 2, 3 amino acid sequences and the selected VL CDRs, 1, 2, 3 amino acid sequences are shown in FIG. 1 (CDRs under Kabat definition), and FIG. 2 (CDRs under Chothia definition).

In some embodiments, the antibody or an antigen-binding fragment described herein can contain a heavy chain variable domain containing one, two, or three of the CDRs of SEQ ID NO: 1 with zero, one or two amino acid insertions, deletions, or substitutions; SEQ ID NO: 2 with zero, one or two amino acid insertions, deletions, or substitutions; SEQ ID NO: 3 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 7 with zero, one or two amino acid insertions, deletions, or substitutions; SEQ ID NO: 8 with zero, one or two amino acid insertions, deletions, or substitutions; SEQ ID NO: 9 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 13 with zero, one or two amino acid insertions, deletions, or substitutions; SEQ ID NO: 14 with zero, one or two amino acid insertions, deletions, or substitutions; SEQ ID NO: 15 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 19 with zero, one or two amino acid insertions, deletions, or substitutions; SEQ ID NO: 20 with zero, one or two amino acid insertions, deletions, or substitutions; SEQ ID NO: 21 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 25 with zero, one or two amino acid insertions, deletions, or substitutions; SEQ ID NO: 26 with zero, one or two amino acid insertions, deletions, or substitutions; SEQ ID NO: 27 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 28 with zero, one or two amino acid insertions, deletions, or substitutions; SEQ ID NO: 29 with zero, one or two amino acid insertions, deletions, or substitutions; SEQ ID NO: 30 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 31 with zero, one or two amino acid insertions, deletions, or substitutions; SEQ ID NO: 32 with zero, one or two amino acid insertions, deletions, or substitutions; SEQ ID NO: 33 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 34 with zero, one or two amino acid insertions, deletions, or substitutions; SEQ ID NO: 35 with zero, one or two amino acid insertions, deletions, or substitutions; SEQ ID NO: 36 with zero, one or two amino acid insertions, deletions, or substitutions.

In some embodiments, the antibody or an antigen-binding fragment described herein can contain a light chain variable domain containing one, two, or three of the CDRs of SEQ ID NO: 4 with zero, one or two amino acid insertions, deletions, or substitutions; SEQ ID NO: 5 with zero, one or two amino acid insertions, deletions, or substitutions; SEQ ID NO: 6 with zero, one or two amino acid insertions, deletions, or substitutions.

In some embodiments, the antibody or an antigen-binding fragment described herein can contain a light chain variable domain containing one, two, or three of the CDRs of SEQ ID NO: 10 with zero, one or two amino acid insertions, deletions, or substitutions; SEQ ID NO: 11 with zero, one or two amino acid insertions, deletions, or substitutions; SEQ ID NO: 12 with zero, one or two amino acid insertions, deletions, or substitutions.

In some embodiments, the antibody or an antigen-binding fragment described herein can contain a light chain variable domain containing one, two, or three of the CDRs of SEQ ID NO: 16 with zero, one or two amino acid insertions, deletions, or substitutions; SEQ ID NO: 17 with zero, one or two amino acid insertions, deletions, or substitutions; SEQ ID NO: 18 with zero, one or two amino acid insertions, deletions, or substitutions.

In some embodiments, the antibody or an antigen-binding fragment described herein can contain a light chain variable domain containing one, two, or three of the CDRs of SEQ ID NO: 22 with zero, one or two amino acid insertions, deletions, or substitutions; SEQ ID NO: 23 with zero, one or two amino acid insertions, deletions, or substitutions; SEQ ID NO: 24 with zero, one or two amino acid insertions, deletions, or substitutions.

The insertions, deletions, and substitutions can be within the CDR sequence, or at one or both terminal ends of the CDR sequence. In some embodiments, the CDR is determined based on Kabat definition scheme. In some embodiments, the CDR is determined based on Chothia definition scheme. In some embodiments, the CDR is determined based on a combination of Kabat and Chothia definition scheme. In some embodiments, the CDR is determined based on IMGT definition. In some embodiments, the CDR is determined based on contact definition.

The disclosure also provides antibodies or antigen-binding fragments thereof that bind to IGF1R. The antibodies or antigen-binding fragments thereof contain a heavy chain variable region (VH) comprising or consisting of an amino acid sequence that is at least 80%, 85%, 90%, or 95% identical to a selected VH sequence, and a light chain variable region (VL) comprising or consisting of an amino acid sequence that is at least 80%, 85%, 90%, or 95% identical to a selected VL sequence. In some embodiments, the selected VH sequence is SEQ ID NO: 37, and the selected VL sequence is SEQ ID NO: 38. In some embodiments, the selected VH sequence is SEQ ID NO: 39 and the selected VL sequence is SEQ ID NO: 40. In some embodiments, the selected VH sequence is SEQ ID NO: 41 and the selected VL sequence is SEQ ID NO: 42. In some embodiments, the selected VH sequence is SEQ ID NO: 43 and the selected VL sequence is SEQ ID NO: 44.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is at least 80% of the length of the reference sequence, and in some embodiments is at least 90%, 95%, or 100%. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For example, the comparison of sequences and determination of percent identity between two sequences can be accomplished using a Blossum 62 scoring matrix with a gap penalty of 12, a gap extend penalty of 4, and a frameshift gap penalty of 5.

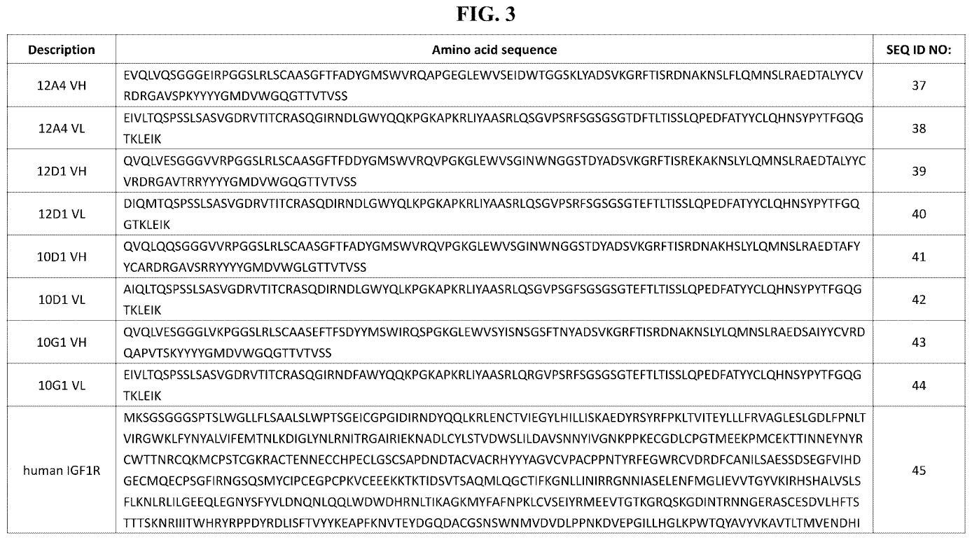

The disclosure also provides nucleic acid comprising a polynucleotide encoding a polypeptide comprising an immunoglobulin heavy chain or an immunoglobulin light chain. The immunoglobulin heavy chain or immunoglobulin light chain comprises CDRs as shown in FIG. 1, or FIG. 2, or have sequences as shown in FIG. 3. When the polypeptides are paired with corresponding polypeptide (e.g., a corresponding heavy chain variable region or a corresponding light chain variable region), the paired polypeptides bind to IGF1R.

The anti-IGF1R antibodies and antigen-binding fragments can also be antibody variants (including derivatives and conjugates) of antibodies or antibody fragments and multi-specific (e.g., bi-specific) antibodies or antibody fragments. Additional antibodies provided herein are polyclonal, monoclonal, multimeric, multi-specific (e.g., bi-specific), human antibodies, chimeric antibodies (e.g., human-mouse chimera), single-chain antibodies, intracellularly-made antibodies (i.e., intrabodies), and antigen-binding fragments thereof. The antibodies or antigen-binding fragments thereof can be of any type (e.g., IgG, IgE, IgM, IgD, IgA, and IgY), class (e.g., IgG1, IgG2, IgG3, IgG4, IgA1, and IgA2), or subclass. In some embodiments, the antibody or antigen-binding fragment thereof is an IgG antibody or antigen-binding fragment thereof.

Fragments of antibodies are suitable for use in the methods provided so long as they retain the desired affinity and specificity of the full-length antibody. Thus, a fragment of an antibody that binds to IGF1R will retain an ability to bind to IGF1R. An Fv fragment is an antibody fragment which contains a complete antigen recognition and binding site. This region consists of a dimer of one heavy and one light chain variable domain in tight association, which can be covalent in nature, for example in scFv. It is in this configuration that the three CDRs of each variable domain interact to define an antigen binding site on the surface of the VH-VL dimer. Collectively, the six CDRs or a subset thereof confer antigen binding specificity to the antibody. However, even a single variable domain (or half of an Fv comprising only three CDRs specific for an antigen) can have the ability to recognize and bind antigen, although usually at a lower affinity than the entire binding site. Single-chain Fv or (scFv) antibody fragments comprise the VH and VL domains (or regions) of antibody, wherein these domains are present in a single polypeptide chain. Generally, the scFv polypeptide further comprises a polypeptide linker between the VH and VL domains, which enables the scFv to form the desired structure for antigen binding.

The present disclosure also provides an antibody or antigen-binding fragment thereof that cross-competes with any antibody or antigen-binding fragment as described herein. The cross-competing assay is known in the art, and is described e.g., in Moore et al., “Antibody cross-competition analysis of the human immunodeficiency virus type 1 gp120 exterior envelope glycoprotein.” Journal of Virology 70.3 (1996): 1863-1872, which is incorporated herein reference in its entirety. In one aspect, the present disclosure also provides an antibody or antigen-binding fragment thereof that binds to the same epitope or region as any antibody or antigen-binding fragment as described herein. The epitope binning assay is known in the art, and is described e.g., in Estep et al. “High throughput solution-based measurement of antibody-antigen affinity and epitope binning.” MAbs. Vol. 5. No. 2. Taylor & Francis, 2013, which is incorporated herein reference in its entirety.

### Antibodies and Antigen Binding Fragments

The present disclosure provides various antibodies and antigen-binding fragments thereof derived from anti-IGF1R antibodies described herein. In general, antibodies (also called immunoglobulins) are made up of two classes of polypeptide chains, light chains and heavy chains. A non-limiting example of antibody of the present disclosure can be an intact, four immunoglobulin chain antibody comprising two heavy chains and two light chains. The heavy chain of the antibody can be of any isotype including IgM, IgG, IgE, IgA, or IgD or sub-isotype including IgG1, IgG2, IgG2a, IgG2b, IgG3, IgG4, IgE1, IgE2, etc. The light chain can be a kappa light chain or a lambda light chain. An antibody can comprise two identical copies of a light chain and two identical copies of a heavy chain. The heavy chains, which each contain one variable domain (or variable region, VH) and multiple constant domains (or constant regions), bind to one another via disulfide bonding within their constant domains to form the “stem” of the antibody. The light chains, which each contain one variable domain (or variable region, VL) and one constant domain (or constant region), each bind to one heavy chain via disulfide binding. The variable region of each light chain is aligned with the variable region of the heavy chain to which it is bound. The variable regions of both the light chains and heavy chains contain three hypervariable regions sandwiched between more conserved framework regions (FR).

These hypervariable regions, known as the complementary determining regions (CDRs), form loops that comprise the antigen binding surface of the antibody. The four framework regions largely adopt a beta-sheet conformation and the CDRs form loops connecting the beta-sheet structure, and in some cases forming part of, the beta-sheet structure. The CDRs in each chain are held in close proximity by the framework regions and, with the CDRs from the other chain, contribute to the formation of the antigen-binding region.

Methods for identifying the CDR regions of an antibody by analyzing the amino acid sequence of the antibody are well known, and a number of definitions of the CDRs are commonly used. The Kabat definition is based on sequence variability, and the Chothia definition is based on the location of the structural loop regions. These methods and definitions are described in, e.g., Martin, “Protein sequence and structure analysis of antibody variable domains,” Antibody engineering, Springer Berlin Heidelberg, 2001. 422-439; Abhinandan, et al. “Analysis and improvements to Kabat and structurally correct numbering of antibody variable domains,” Molecular immunology 45.14 (2008): 3832-3839; Wu, T. T. and Kabat, E. A. (1970) J. Exp. Med. 132:211-250; Martin et al., Methods Enzymol. 203:121-53 (1991); Morea et al., Biophys Chem. 68 (1-3): 9-16 (October 1997); Morea et al., J Mol Biol. 275 (2): 269-94 (January 1998); Chothia et al., Nature 342 (6252): 877-83 (December 1989); Ponomarenko and Bourne, BMC Structural Biology 7:64 (2007); each of which is incorporated herein by reference in its entirety.

The CDRs are important for recognizing an epitope of an antigen. As used herein, an “epitope” is the smallest portion of a target molecule capable of being specifically bound by the antigen binding domain of an antibody. The minimal size of an epitope may be about three, four, five, six, or seven amino acids, but these amino acids need not be in a consecutive linear sequence of the antigen's primary structure, as the epitope may depend on an antigen's three-dimensional configuration based on the antigen's secondary and tertiary structure.

In some embodiments, the antibody is an intact immunoglobulin molecule (e.g., IgG1, IgG2a, IgG2b, IgG3, IgM, IgD, IgE, IgA). The IgG subclasses (IgG1, IgG2, IgG3, and IgG4) are highly conserved, differ in their constant region, particularly in their hinges and upper CH2 domains. The sequences and differences of the IgG subclasses are known in the art, and are described, e.g., in Vidarsson, et al, “IgG subclasses and allotypes: from structure to effector functions.” Frontiers in immunology 5 (2014); Irani, et al. “Molecular properties of human IgG subclasses and their implications for designing therapeutic monoclonal antibodies against infectious diseases.” Molecular immunology 67.2 (2015): 171-182; Shakib, Farouk, ed. The human IgG subclasses: molecular analysis of structure, function and regulation. Elsevier, 2016; each of which is incorporated herein by reference in its entirety.

The antibody can also be an immunoglobulin molecule that is derived from any species (e.g., human, rodent, mouse, camelid). Antibodies disclosed herein also include, but are not limited to, polyclonal, monoclonal, monospecific, polyspecific antibodies, and chimeric antibodies that include an immunoglobulin binding domain fused to another polypeptide. The term “antigen binding domain” or “antigen binding fragment” is a portion of an antibody that retains specific binding activity of the intact antibody, i.e., any portion of an antibody that is capable of specific binding to an epitope on the intact antibody's target molecule. It includes, e.g., Fab, Fab′, F(ab′)₂, and variants of these fragments. Thus, in some embodiments, an antibody or an antigen binding fragment thereof can be, e.g., a scFv, a Fv, a Fd, a dAb, a bispecific antibody, a bispecific scFv, a diabody, a linear antibody, a single-chain antibody molecule, a multi-specific antibody formed from antibody fragments, and any polypeptide that includes a binding domain which is, or is homologous to, an antibody binding domain. Non-limiting examples of antigen binding domains include, e.g., the heavy chain and/or light chain CDRs of an intact antibody, the heavy and/or light chain variable regions of an intact antibody, full length heavy or light chains of an intact antibody, or an individual CDR from either the heavy chain or the light chain of an intact antibody.

Fragments of antibodies are suitable for use in the methods described herein are also provided. The Fab fragment contains a variable and constant domain of the light chain and a variable domain and the first constant domain (CH1) of the heavy chain. F(ab′)₂ antibody fragments comprise a pair of Fab fragments which are generally covalently linked near their carboxy termini by hinge cysteines between them. Other chemical couplings of antibody fragments are also known in the art.

Diabodies are small antibody fragments with two antigen-binding sites, which fragments comprise a VH connected to a VL in the same polypeptide chain (VH and VL). By using a linker that is too short to allow pairing between the two domains on the same chain, the domains are forced to pair with the complementary domains of another chain and create two antigen-binding sites.

Linear antibodies comprise a pair of tandem Fd segments (VH-CH1-VH-CH1) which, together with complementary light chain polypeptides, form a pair of antigen binding regions. Linear antibodies can be bispecific or monospecific.

Antibodies and antibody fragments of the present disclosure can be modified in the Fc region to provide desired effector functions or serum half-life.

Multimerization of antibodies may be accomplished through natural aggregation of antibodies or through chemical or recombinant linking techniques known in the art. For example, some percentage of purified antibody preparations (e.g., purified IgG1 molecules) spontaneously form protein aggregates containing antibody homodimers and other higher-order antibody multimers.

Alternatively, antibody homodimers may be formed through chemical linkage techniques known in the art. For example, heterobifunctional crosslinking agents including, but not limited to SMCC (succinimidyl 4-(maleimidomethyl)cyclohexane-1-carboxylate) and SATA (N-succinimidyl S-acethylthio-acetate) can be used to form antibody multimers. An exemplary protocol for the formation of antibody homodimers is described in Ghetie et al. (Proc. Natl. Acad. Sci. U.S.A. 94:7509-7514, 1997). Antibody homodimers can be converted to Fab′2 homodimers through digestion with pepsin. Another way to form antibody homodimers is through the use of the autophilic T15 peptide described in Zhao et al. (J. Immunol. 25:396-404, 2002).

In some embodiments, the multi-specific antibody is a bi-specific antibody. Bi-specific antibodies can be made by engineering the interface between a pair of antibody molecules to maximize the percentage of heterodimers that are recovered from recombinant cell culture. For example, the interface can contain at least a part of the CH3 domain of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers. This method is described, e.g., in WO 96/27011, which is incorporated by reference in its entirety.

Bi-specific antibodies include cross-linked or “heteroconjugate” antibodies. For example, one of the antibodies in the heteroconjugate can be coupled to avidin and the other to biotin. Heteroconjugate antibodies can also be made using any convenient cross-linking methods. Suitable cross-linking agents and cross-linking techniques are well known in the art and are disclosed in U.S. Pat. No. 4,676,980, which is incorporated herein by reference in its entirety.

Any of the antibodies or antigen-binding fragments described herein may be conjugated to a stabilizing molecule (e.g., a molecule that increases the half-life of the antibody or antigen-binding fragment thereof in a subject or in solution). Non-limiting examples of stabilizing molecules include: a polymer (e.g., a polyethylene glycol) or a protein (e.g., serum albumin, such as human serum albumin). The conjugation of a stabilizing molecule can increase the half-life or extend the biological activity of an antibody or an antigen-binding fragment in vitro (e.g., in tissue culture or when stored as a pharmaceutical composition) or in vivo (e.g., in a human).

In some embodiments, the antibodies or antigen-binding fragments described herein can be conjugated to a therapeutic agent. The antibody-drug conjugate comprising the antibody or antigen-binding fragment thereof can covalently or non-covalently bind to a therapeutic agent. In some embodiments, the therapeutic agent is a cytotoxic or cytostatic agent.

In some embodiments, the antigen binding fragment can form a part of a chimeric antigen receptor (CAR). In some embodiments, the chimeric antigen receptor are fusions of single-chain variable fragments (scFv) as described herein, fused to CD3-zeta transmembrane- and endodomain. In some embodiments, the chimeric antigen receptor also comprises intracellular signaling domains from various costimulatory protein receptors (e.g., CD28, 41BB, ICOS). In some embodiments, the chimeric antigen receptor comprises multiple signaling domains, e.g., CD3z-CD28-41BB or CD3z-CD28-OX40, to increase potency. Thus, in one aspect, the disclosure further provides cells (e.g., T cells) that express the chimeric antigen receptors as described herein.

In some embodiments, the scFv has one heavy chain variable domain, and one light chain variable domain. In some embodiments, the scFv has two heavy chain variable domains, and two light chain variable domains.

In some embodiments, sequences (e.g., CDRs or VH/VL sequences) of the antibody or antigen-binding fragment thereof described herein can be used to generate a bispecific antibody targeting IGF1R and an addition antigen (e.g., CD3, PD-1, PD-L1, HER2 or EGFR).

### Antibody Characteristics

In some embodiments, the antibodies or antigen-binding fragments thereof described herein block the binding between IGF1R and IGF1R ligands (e.g., IGF-1, IGF-2 or insulin (INS)). Thus, by binding to IGF1R, the antibody inhibit IGF1R signaling pathway. In some embodiments, the antibody can reduce tumor volume in an animal. In some embodiments, the antibody can inhibit IGF-1-induced cell proliferation. In some embodiments, the antibody can inhibit IGF1R autophosphorylation. In some embodiments, the antibody can inhibit IGF-1-induced OPN secretion.

In some implementations, the antibody (or antigen-binding fragments thereof) specifically binds to IGF1R (e.g., human IGF1R or monkey IGF1R) with a dissociation rate (k_{off}) of less than 0.1 s⁻¹, less than 0.01 s⁻¹, less than 0.001 s⁻¹, less than 0.0001 s⁻¹, or less than 0.00001 s⁻¹. In some embodiments, the dissociation rate (k_{off}) is greater than 0.01 s⁻¹, greater than 0.001 s⁻¹, greater than 0.0001 s⁻¹, greater than 0.00001 s⁻¹, or greater than 0.000001 s⁻¹.

In some embodiments, kinetic association rates (kₒₙ) is greater than 1×10²/Ms, greater than 1×10³/Ms, greater than 1×10⁴/Ms, greater than 1×10⁵/Ms, or greater than 1×10⁶/Ms. In some embodiments, kinetic association rates (kₒₙ) is less than 1×10⁵/Ms, less than 1×10⁶/Ms, or less than 1×10⁷/Ms.

Affinities can be deduced from the quotient of the kinetic rate constants (KD=K_{off}/kₒₙ). In some embodiments, KD is less than 1×10⁻⁶ M, less than 1×10⁻⁷M, less than 1×10⁻⁸ M, less than 1×10⁻⁹ M, or less than 1×10⁻¹⁰ M. In some embodiments, the KD is less than 50 nM, 30 nM, 20 nM, 15 nM, 10 nM, 9 nM, 8 nM, 7 nM, 6 nM, 5 nM, 4 nM, 3 nM, 2 nM, or 1 nM. In some embodiments, KD is greater than 1× 10-7 M, greater than 1×10⁻⁸ M, greater than 1×10⁻⁹ M, greater than 1×10⁻¹⁰ M, greater than 1× 10-11 M, or greater than 1×10⁻¹² M.

General techniques for measuring the affinity of an antibody for an antigen include, e.g., ELISA, RIA, and surface plasmon resonance (SPR). In some embodiments, the antibody binds to human IGF1R (SEQ ID NO: 45), and/or monkey IGF1R (e.g., SEQ ID NO: 46). In some embodiments, the antibody does not bind to human IGF1R and/or monkey IGF1R. In some embodiments, the antibody does not bind to human insulin receptor INSR (e.g., SEQ ID NO: 47).

In some embodiments, the antibody or antigen-binding fragment thereof described herein has a tumor growth inhibition percentage (TGI_{TV} %) that is greater than 10%, 20%, 30%, 40%, 50%, 60%, 70%, 80%, 90%, 100%, 110%, 120%, 130%, 140%, 150%, 160%, 170%, 180%, 190%, or 200%. In some embodiments, the antibody has a tumor growth inhibition percentage that is less than 60%, 70%, 80%, 90%, 100%, 110%, 120%, 130%, 140%, 150%, 160%, 170%, 180%, 190%, or 200%. The TGI % can be determined, e.g., at 3, 4, 5, 6, 7, 8, 9, 10, 11, 12, 13, 14, 15, 16, 17, 18, 19, 20, 21, 22, 23, 24, 25, 26, 27, 28, 29, or 30 days after the treatment starts, or 1, 2, 3, 4, 5, 6, 7, 8, 9, 10, 11, or 12 months after the treatment starts. As used herein, the tumor growth inhibition percentage (TGI %) is calculated using the following formula:

$$TGI ( \% ) = [ 1 - ( {T}_{i} - {T}_{0} ) / ( {V}_{i} - {V}_{0} ) ] \times 100 \%$$

Ti is the average tumor volume in the treatment group on Day i. T₀ is the average tumor volume in the treatment group on Day zero. Vᵢ is the average tumor volume in the control group on Day i. V₀ is the average tumor volume in the control group on Day zero.

In some embodiments, the antibody or antigen-binding fragment thereof described herein can inhibit IGF-1-induced cell proliferation. In some embodiments, the IGF-1-induced cell proliferation is inhibited by about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99% or 100%.

In some embodiments, the antibody can inhibit IGF1R autophosphorylation. In some embodiments, the IGF1R autophosphorylation is inhibited by about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99% or 100%.

In some embodiments, the antibody can inhibit IGF-1-induced OPN secretion. In some embodiments, the IGF-1-induced OPN secretion is inhibited by about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99% or 100%.

In some embodiments, the antibodies or antigen-binding fragments thereof as described herein are IGF1R antagonist. In some embodiments, the antibodies or antigen-binding fragments thereof as described herein are IGF1R agonist.

In some embodiments, the antibodies or antigen-binding fragments thereof as described herein are not toxic. In some embodiments, no significant differences in body weight can be observed between treatment group and control group e.g., at 3 mg/kg.

In some embodiments, the antibodies or antigen binding fragments can induce complement-dependent cytotoxicity (CDC) and/or antibody dependent cellular cytotoxicity (ADCC), and kill the tumor cell.

In some embodiments, the antibodies or antigen binding fragments have a functional Fc region. In some embodiments, effector function of a functional Fc region is antibody-dependent cell-mediated cytotoxicity (ADCC). In some embodiments, effector function of a functional Fc region is phagocytosis. In some embodiments, effector function of a functional Fc region is ADCC and phagocytosis.

In some embodiments, the antibodies or antigen binding fragments can induce complement-dependent cytotoxicity (CDC).

In some embodiments, the Fc region is human IgG1, human IgG2, human IgG3, or human IgG4. In some embodiments, the antibody is a human IgG1 antibody.

In some embodiments, the antibodies or antigen binding fragments do not have a functional Fc region. For example, the antibodies or antigen binding fragments are Fab, Fab′, F(ab′)₂, and Fv fragments. In some embodiments, the Fc region has LALA mutations (L234A and L235A mutations in EU numbering), or LALA-PG mutations (L234A, L235A, P329G mutations in EU numbering).

In some embodiments, the Fc have a SI mutation (S239D and I332E mutations in EU numbering). In some embodiments, the Fc has one or more of the following mutations M252Y/S254T/T256E according to EU numbering.

In some embodiments, the Fc has one or more of the following mutations T250Q/M428L according to EU numbering.

In some embodiments, the antibodies or antigen binding fragments described herein are stable at high concentration and/or under stressed storage conditions.

In some embodiments, the stability of the antibodies or antigen binding fragments described herein is measured by the relative amount of potential molecular variants, e.g., high molecular weight and/or low molecular weight variants. In some embodiments, the relative amount of potential molecular variants is measured using size exclusion-high-performance liquid chromatography (SEC-HPLC). Other methods of measuring the stability of antibodies are known in the art.

In some embodiments, the potential molecular variant(s) is about 20%, 19%, 18%, 17%, 16%, 15%, 14%, 13%, 12%, 11%, 10%, 9%, 8%, 7%, 6%, 5%, 4%, 3%, 2%, 1% or less of the total sample measured. In some embodiments, the potential molecular variant(s) is not detectable in the sample measured. In some embodiments, the main peak representing the stable antibodies is about 70%, 75%, 80%, 85%, 90%, 95%, 96%, 97%, 98%, 99% or more of the total sample measured.

In some embodiments, the measurement is performed after about 1, 2, 3, 4, 5, 6, or 7 days of storage. In some embodiments, the measurement is performed after about 1, 2, 3, 4, 5, 6, or 7 week(s) of storage. In some embodiments, the measurement is performed after about 4 weeks of storage.

In some embodiments, the antibodies or antigen binding fragments described herein are stored at about 25° C., 30° C., 35° C., 40° C., 45° C., 50° C., 55° C. or more.

In some embodiments, the antibodies or antigen binding fragments described herein are stored at a concentration of at least or about 50 mg/ml, at least or about 70 mg/ml, at least or about 100 mg/ml, at least or about 120 mg/ml, at least or about 150 mg/ml or more. In some embodiments, the antibodies or antigen binding fragments described herein are stored at a concentration of about 50 mg/ml to about 150 mg/ml, about 70 mg/ml to about 150 mg/ml, about 80 mg/ml to about 150 mg/ml, about 90 mg/ml to about 150 mg/ml, about 80 mg/ml to about 120 mg/ml, or about 90 mg/ml to about 110 mg/ml.

### Methods of Making Anti-IGF1R Antibodies

An isolated fragment of human IGF1R (e.g., extracellular region) can be used as an immunogen to generate antibodies using standard techniques for polyclonal and monoclonal antibody preparation. Polyclonal antibodies can be raised in animals by multiple injections (e.g., subcutaneous or intraperitoneal injections) of an antigenic peptide or protein. In some embodiments, the antigenic peptide or protein is injected with at least one adjuvant. In some embodiments, the antigenic peptide or protein can be conjugated to an agent that is immunogenic in the species to be immunized. Animals can be injected with the antigenic peptide or protein more than one time (e.g., twice, three times, or four times).

The full-length polypeptide or protein can be used or, alternatively, antigenic peptide fragments thereof can be used as immunogens. The antigenic peptide of a protein comprises at least 8 (e.g., at least 10, 15, 20, or 30) amino acid residues of the amino acid sequence of IGF1R and encompasses an epitope of the protein such that an antibody raised against the peptide forms a specific immune complex with the protein. As described above, the full length sequence of human IGF1R is known in the art (SEQ ID NO: 45). The full length sequence of monkey IGF1R is known in the art (SEQ ID NO: 46). In some embodiments, a His-tagged human IGF1R protein (ACRO Biosystems Inc., Cat #: IGR-H5229, containing positions 31-932 of SEQ ID NO: 45) is used as the immunogen.

An immunogen typically is used to prepare antibodies by immunizing a suitable subject (e.g., human or transgenic animal expressing at least one human immunoglobulin locus). An appropriate immunogenic preparation can contain, for example, a recombinantly-expressed or a chemically-synthesized polypeptide (e.g., a fragment of human IGF1R). The preparation can further include an adjuvant, such as Freund's complete or incomplete adjuvant, or a similar immunostimulatory agent.

Polyclonal antibodies can be prepared as described above by immunizing a suitable subject with a IGF1R polypeptide, or an antigenic peptide thereof (e.g., part of IGF1R, such as the extracellular region) as an immunogen. The antibody titer in the immunized subject can be monitored over time by standard techniques, such as with an enzyme-linked immunosorbent assay (ELISA) using the immobilized IGF1R polypeptide or peptide. If desired, the antibody molecules can be isolated from the mammal (e.g., from the blood) and further purified by well-known techniques, such as protein A or protein G chromatography to obtain the IgG fraction. At an appropriate time after immunization, e.g., when the specific antibody titers are highest, antibody-producing cells can be obtained from the subject and used to prepare monoclonal antibodies by standard techniques, such as the hybridoma technique originally described by Kohler et al. (Nature 256:495-497, 1975), the human B cell hybridoma technique (Kozbor et al., Immunol. Today 4:72, 1983), the EBV-hybridoma technique (Cole et al., Monoclonal Antibodies and Cancer Therapy, Alan R. Liss, Inc., pp. 77-96, 1985), or trioma techniques. The technology for producing hybridomas is well known (see, generally, Current Protocols in Immunology, 1994, Coligan et al. (Eds.), John Wiley & Sons, Inc., New York, NY). Hybridoma cells producing a monoclonal antibody are detected by screening the hybridoma culture supernatants for antibodies that bind the polypeptide or epitope of interest, e.g., using a standard ELISA assay.

Variants of the antibodies or antigen-binding fragments described herein can be prepared by introducing appropriate nucleotide changes into the DNA encoding a human, humanized, or chimeric antibody, or antigen-binding fragment thereof described herein, or by peptide synthesis. Such variants include, for example, deletions, insertions, or substitutions of residues within the amino acids sequences that make-up the antigen-binding site of the antibody or an antigen-binding domain. In a population of such variants, some antibodies or antigen-binding fragments will have increased affinity for the target protein, e.g., IGF1R. Any combination of deletions, insertions, and/or combinations can be made to arrive at an antibody or antigen-binding fragment thereof that has increased binding affinity for the target. The amino acid changes introduced into the antibody or antigen-binding fragment can also alter or introduce new post-translational modifications into the antibody or antigen-binding fragment, such as changing (e.g., increasing or decreasing) the number of glycosylation sites, changing the type of glycosylation site (e.g., changing the amino acid sequence such that a different sugar is attached by enzymes present in a cell), or introducing new glycosylation sites.

Antibodies disclosed herein can be derived from any species of animal, including mammals. Non-limiting examples of native antibodies include antibodies derived from humans, primates, e.g., monkeys and apes, cows, pigs, horses, sheep, camelids (e.g., camels and llamas), chicken, goats, and rodents (e.g., rats, mice, hamsters and rabbits), including transgenic rodents genetically engineered to produce human antibodies.

Human and humanized antibodies include antibodies having variable and constant regions derived from (or having the same amino acid sequence as those derived from) human germline immunoglobulin sequences. Human antibodies may include amino acid residues not encoded by human germline immunoglobulin sequences (e.g., mutations introduced by random or site-specific mutagenesis in vitro or by somatic mutation in vivo), for example in the CDRs.

A humanized antibody, typically has a human framework (FR) grafted with non-human CDRs. Thus, a humanized antibody has one or more amino acid sequence introduced into it from a source which is non-human. These non-human amino acid residues are often referred to as “import” residues, which are typically taken from an “import” variable domain. Humanization can be essentially performed by e.g., substituting rodent CDRs or CDR sequences for the corresponding sequences of a human antibody. These methods are described in e.g., Jones et al. “Replacing the complementarity-determining regions in a human antibody with those from a mouse.” Nature 321.6069 (1986): 522; Riechmann et al. “Reshaping human antibodies for therapy.” Nature 332.6162 (1988): 323; Dall'Acqua et al. “Antibody humanization by framework shuffling.” Methods 36.1 (2005): 43-60; each of which is incorporated by reference herein in its entirety. Accordingly, “humanized” antibodies are chimeric antibodies wherein substantially less than an intact human V domain has been substituted by the corresponding sequence from a non-human species. In practice, humanized antibodies are typically mouse antibodies in which some CDR residues and some FR residues are substituted by residues from analogous sites in human antibodies.

The choice of human VH and VL domains to be used in making the humanized antibodies is very important for reducing immunogenicity. According to the so-called “best-fit” method, the sequence of the V domain of a mouse antibody is screened against the entire library of known human-domain sequences. The human sequence which is closest to that of the mouse is then accepted as the human FR for the humanized antibody (Sims et al. “A humanized CD18 antibody can block function without cell destruction.” The Journal of Immunology 151.4 (1993): 2296-2308; Chothia, et al., “Canonical structures for the hypervariable regions of immunoglobulins.” Journal of Molecular Biology 196.4 (1987): 901-917).

It is further important that antibodies be humanized with retention of high specificity and affinity for the antigen and other favorable biological properties. To achieve this goal, humanized antibodies can be prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved.

Ordinarily, amino acid sequence variants of the human, humanized, or chimeric anti-IGF1R antibody will contain an amino acid sequence having at least 75%, 80%, 85%, 90%, 95%, 96%, 97%, 98%, or 99% percent identity with a sequence present in the light or heavy chain of the original antibody.

In some embodiments, a mouse (e.g., RenMab mouse) with a humanized heavy chain immunoglobulin locus and a humanized kappa chain immunoglobulin locus is used to generate antibodies. The heavy chain immunoglobulin locus is a region on the chromosome that contains genes for the heavy chains of antibodies. The locus can include e.g., human IGHV (variable) genes, human IGHD (diversity) genes, human IGHJ (joining) genes, and mouse heavy chain constant domain genes. The kappa chain immunoglobulin locus is a region on the chromosome that contains genes that encode the light chains of antibodies (kappa chain). The kappa chain immunoglobulin locus can include e.g., human IGKV (variable) genes, human IGKJ (joining) genes, and mouse light chain constant domain genes. A detailed description regarding RenMab mice can be found in PCT/CN2020/075698, which is incorporated herein by reference in its entirety. The antibodies generated by the mice have a full human VH, a full human VL, and mouse constant regions. In some embodiments, the human VH and human VL is linked to a human IgG constant regions (e.g., IgG1, IgG2, IgG3, and IgG4).

In some embodiments, a mouse (e.g., RenLite™ mouse) with a humanized heavy chain immunoglobulin locus and a humanized kappa chain immunoglobulin locus is used to generate antibodies. The heavy chain immunoglobulin locus is a region on the chromosome that contains genes for the heavy chains of antibodies. The locus can include e.g., human IGHV (variable) genes, human IGHD (diversity) genes, human IGHJ (joining) genes, and mouse heavy chain constant domain genes. The kappa chain immunoglobulin locus is a region on the chromosome that contains genes that encode a common light chains. The kappa chain immunoglobulin locus can include e.g., a human IGKV (variable) gene, a human IGKJ (joining) gene, and mouse light chain constant domain genes. A detailed description regarding RenLite™ mice can be found in PCT/CN2021/097652, which is incorporated herein by reference in its entirety.

Identity or homology with respect to an original sequence is usually the percentage of amino acid residues present within the candidate sequence that are identical with a sequence present within the human, humanized, or chimeric anti-IGF1R antibody or fragment, after aligning the sequences and introducing gaps, if necessary, to achieve the maximum percent sequence identity, and not considering any conservative substitutions as part of the sequence identity.

Additional modifications to the anti-IGF1R antibodies or antigen-binding fragments can be made. For example, a cysteine residue(s) can be introduced into the Fc region, thereby allowing interchain disulfide bond formation in this region. The homodimeric antibody thus generated may have any increased half-life in vitro and/or in vivo. Homodimeric antibodies with increased half-life in vitro and/or in vivo can also be prepared using heterobifunctional cross-linkers as described, for example, in Wolff et al. Wolff et al. (“Monoclonal antibody homodimers: enhanced antitumor activity in nude mice.” Cancer research 53.11 (1993): 2560-2565). Alternatively, an antibody can be engineered which has dual Fc regions.

In some embodiments, a covalent modification can be made to the anti-IGF1R antibody or antigen-binding fragment thereof. These covalent modifications can be made by chemical or enzymatic synthesis, or by enzymatic or chemical cleavage. Other types of covalent modifications of the antibody or antibody fragment are introduced into the molecule by reacting targeted amino acid residues of the antibody or fragment with an organic derivatization agent that is capable of reacting with selected side chains or the N- or C-terminal residues.

In some embodiments, antibody variants are provided having a carbohydrate structure that lacks fucose attached (directly or indirectly) to an Fc region. For example, the amount of fucose in such antibody composition may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues; or position 314 in Kabat numbering); however, Asn297 may also be located about ±3 amino acids upstream or downstream of position 297, i.e., between positions 294 and 300, due to minor sequence variations in antibodies. Such fucosylation variants may have improved ADCC function. In some embodiments, to reduce glycan heterogeneity, the Fc region of the antibody can be further engineered to replace the Asparagine at position 297 with Alanine (N297A).

In some embodiments, to facilitate production efficiency by avoiding Fab-arm exchange, the Fc region of the antibodies was further engineered to replace the serine at position 228 (EU numbering) of IgG4 with proline (S228P). A detailed description regarding S228 mutation is described, e.g., in Silva et al. “The S228P mutation prevents in vivo and in vitro IgG4 Fab-arm exchange as demonstrated using a combination of novel quantitative immunoassays and physiological matrix preparation.” Journal of Biological Chemistry 290.9 (2015): 5462-5469, which is incorporated by reference in its entirety.

### Recombinant Vectors

The present disclosure also provides recombinant vectors (e.g., an expression vectors) that include an isolated polynucleotide disclosed herein (e.g., a polynucleotide that encodes a polypeptide disclosed herein), host cells into which are introduced the recombinant vectors (i.e., such that the host cells contain the polynucleotide and/or a vector comprising the polynucleotide), and the production of recombinant antibody polypeptides or fragments thereof by recombinant techniques.

As used herein, a “vector” is any construct capable of delivering one or more polynucleotide(s) of interest to a host cell when the vector is introduced to the host cell. An “expression vector” is capable of delivering and expressing the one or more polynucleotide(s) of interest as an encoded polypeptide in a host cell into which the expression vector has been introduced. Thus, in an expression vector, the polynucleotide of interest is positioned for expression in the vector by being operably linked with regulatory elements such as a promoter, enhancer, and/or a poly-A tail, either within the vector or in the genome of the host cell at or near or flanking the integration site of the polynucleotide of interest such that the polynucleotide of interest will be translated in the host cell introduced with the expression vector.

A vector can be introduced into the host cell by methods known in the art, e.g., electroporation, chemical transfection (e.g., DEAE-dextran), transformation, transfection, and infection and/or transduction (e.g., with recombinant virus). Thus, non-limiting examples of vectors include viral vectors (which can be used to generate recombinant virus), naked DNA or RNA, plasmids, cosmids, phage vectors, and DNA or RNA expression vectors associated with cationic condensing agents.

In some implementations, a polynucleotide disclosed herein (e.g., a polynucleotide that encodes a polypeptide disclosed herein) is introduced using a viral expression system (e.g., vaccinia or other pox virus, retrovirus, or adenovirus), which may involve the use of a non-pathogenic (defective), replication competent virus, or may use a replication defective virus. In the latter case, viral propagation generally will occur only in complementing virus packaging cells. Suitable systems are disclosed, for example, in Fisher-Hoch et al., 1989, Proc. Natl. Acad. Sci. USA 86:317-321; Flexner et al., 1989, Ann. N.Y. Acad Sci. 569:86-103; Flexner et al., 1990, Vaccine, 8:17-21; U.S. Pat. Nos. 4,603,112, 4,769,330, and 5,017,487; WO 89/01973; U.S. Pat. No. 4,777,127; GB 2,200,651; EP 0,345,242; WO 91/02805; Berkner-Biotechniques, 6:616-627, 1988; Rosenfeld et al., 1991, Science, 252:431-434; Kolls et al., 1994, Proc. Natl. Acad. Sci. USA, 91:215-219; Kass-Eisler et al., 1993, Proc. Natl. Acad. Sci. USA, 90:11498-11502; Guzman et al., 1993, Circulation, 88:2838-2848; and Guzman et al., 1993, Cir. Res., 73:1202-1207. Techniques for incorporating DNA into such expression systems are well known to those of ordinary skill in the art. The DNA may also be “naked,” as described, for example, in Ulmer et al., 1993, Science, 259:1745-1749, and Cohen, 1993, Science, 259:1691-1692. The uptake of naked DNA may be increased by coating the DNA onto biodegradable beads that are efficiently transported into the cells.

For expression, the DNA insert comprising an antibody-encoding or polypeptide-encoding polynucleotide disclosed herein can be operatively linked to an appropriate promoter (e.g., a heterologous promoter), such as the phage lambda PL promoter, the E. coli lac, trp and tac promoters, the SV40 early and late promoters and promoters of retroviral LTRs, to name a few. Other suitable promoters are known to the skilled artisan. In some embodiments, the promoter is a cytomegalovirus (CMV) promoter. The expression constructs can further contain sites for transcription initiation, termination and, in the transcribed region, a ribosome binding site for translation. The coding portion of the mature transcripts expressed by the constructs may include a translation initiating at the beginning and a termination codon (UAA, UGA, or UAG) appropriately positioned at the end of the polypeptide to be translated.

As indicated, the expression vectors can include at least one selectable marker. Such markers include dihydrofolate reductase or neomycin resistance for eukaryotic cell culture and tetracycline or ampicillin resistance genes for culturing in E. coli and other bacteria. Representative examples of appropriate hosts include, but are not limited to, bacterial cells, such as E. coli, Streptomyces, and Salmonella typhimurium cells; fungal cells, such as yeast cells; insect cells such as Drosophila S2 and Spodoptera Sf9 cells; animal cells such as CHO, COS, Bowes melanoma, and HK 293 cells; and plant cells. Appropriate culture mediums and conditions for the host cells described herein are known in the art.

Non-limiting vectors for use in bacteria include pQE70, pQE60 and pQE-9, available from Qiagen; pBS vectors, Phagescript vectors, Bluescript vectors, pNH8A, pNH16a, pNH18A, pNH46A, available from Stratagene; and ptrc99a, pKK223-3, pKK233-3, pDR540, pRIT5 available from Pharmacia. Non-limiting eukaryotic vectors include pWLNEO, pSV2CAT, pOG44, pXT1 and pSG available from Stratagene; and pSVK3, pBPV, pMSG and pSVL available from Pharmacia. Other suitable vectors will be readily apparent to the skilled artisan.

Non-limiting bacterial promoters suitable for use include the E. coli lacI and lacZ promoters, the T3 and T7 promoters, the gpt promoter, the lambda PR and PL promoters and the trp promoter. Suitable eukaryotic promoters include the CMV immediate early promoter, the HSV thymidine kinase promoter, the early and late SV40 promoters, the promoters of retroviral LTRs, such as those of the Rous sarcoma virus (RSV), and metallothionein promoters, such as the mouse metallothionein-I promoter.

In the yeast Saccharomyces cerevisiae, a number of vectors containing constitutive or inducible promoters such as alpha factor, alcohol oxidase, and PGH can be used.

Introduction of the construct into the host cell can be effected by calcium phosphate transfection, DEAE-dextran mediated transfection, cationic lipid-mediated transfection, electroporation, transduction, infection or other methods. Such methods are described in many standard laboratory manuals, such as Davis et al., Basic Methods in Molecular Biology (1986), which is incorporated herein by reference in its entirety.

Transcription of DNA encoding an antibody of the present disclosure by higher eukaryotes may be increased by inserting an enhancer sequence into the vector. Enhancers are cis-acting elements of DNA, usually about from 10 to 300 bp that act to increase transcriptional activity of a promoter in a given host cell-type. Examples of enhancers include the SV40 enhancer, which is located on the late side of the replication origin at base pairs 100 to 270, the cytomegalovirus early promoter enhancer, the polyoma enhancer on the late side of the replication origin, and adenovirus enhancers.

For secretion of the translated protein into the lumen of the endoplasmic reticulum, into the periplasmic space or into the extracellular environment, appropriate secretion signals may be incorporated into the expressed polypeptide. The signals may be endogenous to the polypeptide or they may be heterologous signals.

The polypeptide (e.g., antibody) can be expressed in a modified form, such as a fusion protein (e.g., a GST-fusion) or with a histidine-tag, and may include not only secretion signals, but also additional heterologous functional regions. For instance, a region of additional amino acids, particularly charged amino acids, may be added to the N-terminus of the polypeptide to improve stability and persistence in the host cell, during purification, or during subsequent handling and storage. Also, peptide moieties can be added to the polypeptide to facilitate purification. Such regions can be removed prior to final preparation of the polypeptide. The addition of peptide moieties to polypeptides to engender secretion or excretion, to improve stability and to facilitate purification, among others, are familiar and routine techniques in the art.

### Methods of Treatment

The antibodies or antigen-binding fragments thereof of the present disclosure can be used for various therapeutic purposes.

In one aspect, the disclosure provides methods for treating a cancer in a subject, methods of reducing the rate of the increase of volume of a tumor in a subject over time, methods of reducing the risk of developing a metastasis, or methods of reducing the risk of developing an additional metastasis in a subject. In some embodiments, the treatment can halt, slow, retard, or inhibit progression of a cancer. In some embodiments, the treatment can result in the reduction of in the number, severity, and/or duration of one or more symptoms of the cancer in a subject.

In one aspect, the disclosure provides methods for treating an eye disease (e.g., a thyroid eye disease (TED) or thyroid-associated ophthalmopathy) in a subject, methods of reducing the rate of the progression of the disease in a subject over time, methods of reducing the risk of developing the eye disease in a subject. In some embodiments, the treatment can halt, slow, retard, or inhibit progression of an eye disease. In some embodiments, the treatment can result in the reduction of in the number, severity, and/or duration of one or more symptoms of the eye disease in a subject.

In some embodiments, the methods for treating the eye disease described herein is combined with one or more additional therapies. Examples of the therapies include, but are not limited to eyelid surgery, eye muscle surgery, and orbital decompression surgery.

In one aspect, the disclosure provides methods for treating a bone disease in a subject, methods of reducing the rate of the progression of the disease in a subject over time, methods of reducing the risk of developing the bone disease in a subject. In some embodiments, the treatment can halt, slow, retard, or inhibit progression of a bone disease. In some embodiments, the treatment can result in the reduction of in the number, severity, and/or duration of one or more symptoms of the bone disease in a subject.

In one aspect, the disclosure provides methods for treating an IGF1R-related disease in a subject, methods of reducing the rate of the progression of the disease in a subject over time, methods of reducing the risk of developing the disease in a subject. In some embodiments, the treatment can halt, slow, retard, or inhibit progression of the IGF1R-related disease. In some embodiments, the treatment can result in the reduction of in the number, severity, and/or duration of one or more symptoms of the IGF1R-related disease in a subject. As used herein, the IGF1R-related disease is a disease that is caused or relate to the activation of the IGF1R signaling pathway. In some embodiments, the IGF1R-related disease is a cancer. In some embodiments, the IGF1R-related disease is an eye disease (e.g., a thyroid eye disease (TED) or thyroid-associated ophthalmopathy). In some embodiments, the IGF1R-related disease is a bone disease.

In one aspect, the disclosure features methods that include administering a therapeutically effective amount of an antibody or antigen-binding fragment thereof disclosed herein to a subject in need thereof (e.g., a subject having, or identified or diagnosed as having, a cancer), e.g., lung cancer, non-small cell lung carcinoma (NSCLC), small cell lung cancer (SCLC). In some embodiments, the subject has a solid tumor or advanced solid tumors.

In some embodiments, the anti-IGF1R antibody is designed for treating non-small cell lung carcinoma (NSCLC), and/or small cell lung cancer (SCLC).

In some embodiments, the cancer is non-small cell lung cancer.

In some embodiments, the compositions and methods disclosed herein can be used for treatment of patients at risk for a cancer. Patients with cancer can be identified with various methods known in the art.

In some aspects, the disclosure relates to a method of treating an autoimmune disease or inflammation, the method comprising administering to the subject an effective amount of a composition comprising the antibody or antigen-binding fragment thereof as described herein or the antibody-drug conjugate as described herein.

In one aspect, the disclosure provides methods for treating, preventing, or reducing the risk of developing disorders associated with an abnormal or unwanted immune response, e.g., an autoimmune disorder. These autoimmune disorders include, but are not limited to Grave's disease. In some embodiments, the antibodies or antigen binding fragments can be used to treat inflammation. In some embodiments, the anti-IGF1R antibody is designed for treating other diseases or disorders, e.g., microbial infection and allergic disorders.

In some aspects, the disclosure relates to a method of inhibiting immune response in a subject, the method comprising administering to the subject an effective amount of a composition comprising the antibody or antigen-binding fragment thereof as described herein or the antibody-drug conjugate as described herein. In some embodiments, the subject has an autoimmune disease.

As used herein, by an “effective amount” is meant an amount or dosage sufficient to effect beneficial or desired results including halting, slowing, retarding, or inhibiting progression of a disease, e.g., a cancer. An effective amount will vary depending upon, e.g., an age and a body weight of a subject to which the antibody, antigen binding fragment, antibody-encoding polynucleotide, vector comprising the polynucleotide, and/or compositions thereof is to be administered, a severity of symptoms and a route of administration, and thus administration can be determined on an individual basis.

An effective amount can be administered in one or more administrations. By way of example, an effective amount of an antibody or an antigen binding fragment is an amount sufficient to ameliorate, stop, stabilize, reverse, inhibit, slow and/or delay progression of an autoimmune disease or a cancer in a patient or is an amount sufficient to ameliorate, stop, stabilize, reverse, slow and/or delay proliferation of a cell (e.g., a biopsied cell, any of the cancer cells described herein, or cell line (e.g., a cancer cell line)) in vitro. As is understood in the art, an effective amount of an antibody or antigen binding fragment may vary, depending on, inter alia, patient history as well as other factors such as the type (and/or dosage) of antibody used.

Effective amounts and schedules for administering the antibodies, antibody-encoding polynucleotides, and/or compositions disclosed herein may be determined empirically, and making such determinations is within the skill in the art. Those skilled in the art will understand that the dosage that must be administered will vary depending on, for example, the mammal that will receive the antibodies, antibody-encoding polynucleotides, and/or compositions disclosed herein, the route of administration, the particular type of antibodies, antibody-encoding polynucleotides, antigen binding fragments, and/or compositions disclosed herein used and other drugs being administered to the mammal.

A typical daily dosage of an effective amount of an antibody is 1 mg/kg to 10 mg/kg (mg per kg of patient weight).

In any of the methods described herein, the at least one antibody, antigen-binding fragment thereof, or pharmaceutical composition (e.g., any of the antibodies, antigen-binding fragments, or pharmaceutical compositions described herein) and, optionally, at least one additional therapeutic agent can be administered to the subject. In some embodiments, at least two different antibodies and/or antigen-binding fragments are administered in the same composition (e.g., a liquid composition). In some embodiments, at least one antibody or antigen-binding fragment and at least one additional therapeutic agent are administered in the same composition (e.g., a liquid composition). In some embodiments, the at least one antibody or antigen-binding fragment and the at least one additional therapeutic agent are administered in two different compositions (e.g., a liquid composition containing at least one antibody or antigen-binding fragment and a solid oral composition containing at least one additional therapeutic agent). In some embodiments, the at least one additional therapeutic agent is administered as a pill, tablet, or capsule. In some embodiments, the at least one additional therapeutic agent is administered in a sustained-release oral formulation.

In some embodiments, the one or more additional therapeutic agents can be administered to the subject prior to, or after administering the at least one antibody, antigen-binding antibody fragment, or pharmaceutical composition (e.g., any of the antibodies, antigen-binding antibody fragments, or pharmaceutical compositions described herein). In some embodiments, the one or more additional therapeutic agents and the at least one antibody, antigen-binding antibody fragment, or pharmaceutical composition (e.g., any of the antibodies, antigen-binding antibody fragments, or pharmaceutical compositions described herein) are administered to the subject such that there is an overlap in the bioactive period of the one or more additional therapeutic agents and the at least one antibody or antigen-binding fragment (e.g., any of the antibodies or antigen-binding fragments described herein) in the subject.

In some embodiments, the subject can be administered the at least one antibody, antigen-binding antibody fragment, or pharmaceutical composition (e.g., any of the antibodies, antigen-binding antibody fragments, or pharmaceutical compositions described herein) over an extended period of time. A skilled medical professional may determine the length of the treatment period using any of the methods described herein for diagnosing or following the effectiveness of treatment (e.g., the observation of at least one symptom of cancer). As described herein, a skilled medical professional can also change the identity and number (e.g., increase or decrease) of antibodies or antigen-binding antibody fragments (and/or one or more additional therapeutic agents) administered to the subject and can also adjust (e.g., increase or decrease) the dosage or frequency of administration of at least one antibody or antigen-binding antibody fragment (and/or one or more additional therapeutic agents) to the subject based on an assessment of the effectiveness of the treatment (e.g., using any of the methods described herein and known in the art).

In some embodiments, one or more additional therapeutic agents can be administered to the subject. In some embodiments, the additional therapeutic agent is an anti-CD3 antibody, an anti-PD-1 antibody, an anti-PD-L1 antibody, an anti-HER2 antibody or an anti-EGFR antibody.

### Pharmaceutical Compositions and Routes of Administration

Also provided herein are pharmaceutical compositions that contain at least one (e.g., one, two, three, or four) of the antibodies or antigen-binding fragments described herein. Two or more (e.g., two, three, or four) of any of the antibodies or antigen-binding fragments described herein can be present in a pharmaceutical composition in any combination. The pharmaceutical compositions may be formulated in any manner known in the art.

Pharmaceutical compositions are formulated to be compatible with their intended route of administration (e.g., intravenous, intraarterial, intramuscular, intradermal, subcutaneous, or intraperitoneal). The compositions can include a sterile diluent (e.g., sterile water or saline), a fixed oil, or other synthetic solvents, antibacterial or antifungal agents and the like, antioxidants, such as chelating agents, buffers, and isotonic agents, such as sugars, polyalcohols, or salts, or any combination thereof. Liposomal suspensions can also be used as pharmaceutically acceptable carriers. Preparations of the compositions can be formulated and enclosed in ampules, disposable syringes, or multiple dose vials. Where required (as in, for example, injectable formulations), proper fluidity can be maintained by, for example, the use of a coating, such as lecithin, or a surfactant. Absorption of the antibody or antigen-binding fragment thereof can be prolonged by including an agent that delays absorption. Alternatively, controlled release can be achieved by implants and microencapsulated delivery systems, which can include biodegradable, biocompatible polymers.

Compositions containing one or more of any of the antibodies or antigen-binding fragments described herein can be formulated for parenteral (e.g., intravenous, intraarterial, intramuscular, intradermal, subcutaneous, or intraperitoneal) administration in dosage unit form (i.e., physically discrete units containing a predetermined quantity of active compound for ease of administration and uniformity of dosage).

Pharmaceutical compositions for parenteral administration are preferably sterile and substantially isotonic and manufactured under Good Manufacturing Practice (GMP) conditions. Pharmaceutical compositions can be provided in unit dosage form (i.e., the dosage for a single administration). Pharmaceutical compositions can be formulated using one or more physiologically acceptable carriers, diluents, excipients or auxiliaries. The formulation depends on the route of administration chosen. For injection, antibodies can be formulated in aqueous solutions, preferably in physiologically-compatible buffers to reduce discomfort at the site of injection. The solution can contain formulatory agents such as suspending, stabilizing and/or dispersing agents. Alternatively, antibodies can be in lyophilized form for constitution with a suitable vehicle, e.g., sterile pyrogen-free water, before use.

Toxicity and therapeutic efficacy of compositions can be determined by standard pharmaceutical procedures in cell cultures or experimental animals (e.g., monkeys). One can, for example, determine the LD50 (the dose lethal to 50% of the population) and the ED50 (the dose therapeutically effective in 50% of the population): the therapeutic index being the ratio of LD50:ED50. Agents that exhibit high therapeutic indices are preferred. Where an agent exhibits an undesirable side effect, care should be taken to minimize potential damage (i.e., reduce unwanted side effects). Toxicity and therapeutic efficacy can be determined by other standard pharmaceutical procedures.

Data obtained from cell culture assays and animal studies can be used in formulating an appropriate dosage of any given agent for use in a subject (e.g., a human). A therapeutically effective amount of the one or more (e.g., one, two, three, or four) antibodies or antigen-binding fragments thereof (e.g., any of the antibodies or antibody fragments described herein) will be an amount that treats the disease (e.g., kills cancer cells) in a subject (e.g., a human subject identified as having cancer), or a subject identified as being at risk of developing the disease (e.g., a subject who has previously developed cancer but now has been cured), decreases the severity, frequency, and/or duration of one or more symptoms of a disease in a subject (e.g., a human). The effectiveness and dosing of any of the antibodies or antigen-binding fragments described herein can be determined by a health care professional or veterinary professional using methods known in the art, as well as by the observation of one or more symptoms of disease in a subject (e.g., a human). Certain factors may influence the dosage and timing required to effectively treat a subject (e.g., the severity of the disease or disorder, previous treatments, the general health and/or age of the subject, and the presence of other diseases).

Exemplary doses include milligram or microgram amounts of any of the antibodies or antigen-binding fragments described herein per kilogram of the subject's weight (e.g., about 1 mg/kg to about 10 mg/kg). While these doses cover a broad range, one of ordinary skill in the art will understand that therapeutic agents, including antibodies and antigen-binding fragments thereof, vary in their potency, and effective amounts can be determined by methods known in the art. Typically, relatively low doses are administered at first, and the attending health care professional or veterinary professional (in the case of therapeutic application) or a researcher (when still working at the development stage) can subsequently and gradually increase the dose until an appropriate response is obtained. In addition, it is understood that the specific dose level for any particular subject will depend upon a variety of factors including the activity of the specific compound employed, the age, body weight, general health, gender, and diet of the subject, the time of administration, the route of administration, the rate of excretion, and the half-life of the antibody or antibody fragment in vivo.

The pharmaceutical compositions can be included in a container, pack, or dispenser together with instructions for administration. The disclosure also provides methods of manufacturing the antibodies or antigen binding fragments thereof for various uses as described herein.

### EXAMPLES

The invention is further described in the following examples, which do not limit the scope of the invention described in the claims.

### Example 1. Generating Anti-IGF1R Antibody

To generate antibodies against human IGF1R, RenMab mice were immunized with His-tagged human IGF1R protein (ACRO Biosystems Inc., Cat #: IGR-H5229, containing positions 31-932 of SEQ ID NO: 45). The antibody immune response was monitored by an antigen-specific immunoassay.

RenMab mice have both a humanized heavy chain immunoglobulin locus and a humanized kappa chain immunoglobulin locus. The heavy chain immunoglobulin locus is a region on the chromosome that contains genes for the heavy chains of antibodies. The locus includes IGHV (variable), IGHD (diversity), IGHJ (joining), and heavy chain constant domain genes. The kappa chain immunoglobulin locus is a region on the chromosome that contains genes that encode the light chains of antibodies (kappa chain). The kappa chain immunoglobulin locus includes IGKV (variable), IGKJ (joining), and light chain constant domain genes. A detailed description regarding RenMab mice can be found in PCT/CN2020/075698, which is incorporated herein by reference in its entirety.

A total of three immunizations were performed. The immunizations were separated by two weeks. One week after the last immunization, retro-orbital blood was collected, and the antibody titer in serum was determined by Fluorescence-Activated Cell Sorting (FACS). Mice with high titer were selected two weeks later for impulse immunizations. His-tagged human IGF1R protein or CHO-S cells expressing human IGF1R protein were used for impulse immunizations by intraperitoneal injection and tail vein injection, respectively.

When a desired immune response was achieved, antigen-specific immune cells were isolated from the immunized mice to further obtain anti-IGF1R antibodies or to obtain the light chain and heavy chain variable region sequences of the anti-IGF1R antibodies. For example, single cell technology (for example, using Beacon® Optofluidic System, Berkeley Lights Inc.) was used to screen and find plasma cells that secrete antigen-specific monoclonal antibodies, and reverse transcription and PCR sequencing were used to obtain antibody variable region sequences. The obtained variable region sequences were cloned into a vector containing a sequence encoding the human IgG1 constant region for antibody expression. The binding activity of the expressed antibody to IGF1R was verified using FACS. Exemplary antibodies obtained included: 12A4, 12D1, 10D1 and 10G1.

The VH and VL CDR 1-3 sequences of 12A4, 12D1, 10D1 and 10G1 are shown in FIG. 1 or FIG. 2, and their VH and the VL regions are shown in FIG. 3.

The constant region can also include some other mutations. For example, when YTE mutations (M252Y/S254T/T256E according to EU numbering) are introduced into the Fc region of 12A4, the resulting antibody is named as 12A4-YTE. When QL mutations (T250Q/M428L according to EU numbering) are introduced into the Fc region of 12A4, the resulting antibody is named as 12A4-QL. The sequences of light chain constant region, the heavy chain constant region with mutations are shown in SEQ ID NOs: 50-53 respectively.

### Example 2. Binding Activity of Anti-IGF1R Antibodies

INSR (insulin receptor) and IGF1R are both members of the superfamily of receptor tyrosine kinases (RTKs). The binding activity of anti-IGF1R antibodies to human IGF1R, human INSR and monkey IGF1R was verified by flow cytometry. Briefly, CHO-S-hIGF1R cells (expressing human IGF1R (SEQ ID NO: 45), CHO-S-fasIGF1R cells (expressing cynomolgus monkey IGF1R (SEQ ID NO: 46) and CHO-S-hINSR (expressing SEQ ID NO: 47) were plated in a 96-well plate at a density of 2×10⁵ cells/well respectively. Purified anti-IGF1R antibody with a concentration of 20 μg/mL was added to each well and was incubated at 4° C. for 30 minutes. Then, after one wash with PBS, the cells were incubated with the secondary antibody Alexa Fluor® 647 anti-human IgG Fcγ (Jackson ImmunoResearch Laboratories, Inc., Cat #: 109-606-170) at 4° C. for 15 minutes before flow cytometry analysis. Human IgG1 was used as ISO control. The results are shown in the table below.

TABLE 1

| | | Percentage of |
|---|---|---|
| Antibody | Cells | positive cells |
| ISO | CHO-S-hIGF1R | 0.7% |
| | CHO-S-fasIGF1R | 0.3% |
| | CHO-S-hINSR | 0.4% |
| Teprotumumab | CHO-S-hIGF1R | 45.5% |
| analog | CHO-S-fasIGF1R | 73.0% |
| | CHO-S-hINSR | 1.5% |
| 12A4 | CHO-S-hIGF1R | 50.3% |
| | CHO-S-fasIGF1R | 54.4% |
| | CHO-S-hINSR | 0.4% |
| 12D1 | CHO-S-hIGF1R | 39.3% |
| | CHO-S-fasIGF1R | 60.7% |
| | CHO-S-hINSR | 0.5% |
| 10D1 | CHO-S-hIGF1R | 36.5% |
| | CHO-S-fasIGF1R | 64.8% |
| | CHO-S-hINSR | 1.5% |
| 10G1 | CHO-S-hIGF1R | 46.4% |
| | CHO-S-fasIGF1R | 73.9% |
| | CHO-S-hINSR | 0.7% |

Teprotumumab is a human IgG1 monoclonal antibody targeting IGF1R developed by Genmab in collaboration with River Vision (now Horizon Pharma) for the treatment of Graves' Orbitopathy and scleroderma. The VH and VL sequences of Teprotumumab are shown in SEQ ID NO: 48 and SEQ ID NO: 49, respectively.

The results showed that all four antibodies 12A4, 12D1, 10D1 and 10G1 exhibited binding activities to human and monkey IGF1R, and none of them showed binding activity to human INSR.

### Example 3. Binding Affinity of Anti-IGF1R Antibodies

The affinity of the anti-IGF1R antibodies to His-tagged human IGF1R protein (hIGF1R-His, ACRO Biosystems Inc., Cat #: IGR-H5229) and His-tagged cynomolgus monkey IGF1R protein (cIGF1R-His, ACRO Biosystems Inc., Cat #: IGR-C5225) were measured using surface plasmon resonance (SPR) using Biacore™ (Biacore, Inc., Piscataway N.J.) 8K biosensor equipped with pre-immobilized Protein A sensor chips.

Purified anti-IGF1R antibodies were captured on the Protein A chip (Series S Sensor Chip Protein A) for the detection. 1 μg/mL or 10 μg/mL purified anti-IGF1R antibodies was loaded at 10 μL/min to bind to a gradient concentration of recombinant hIGF1R-His or cIGF1R-His (400 nM, 200 nM, 100 nM, 50 nM, 25 nM, 12.5 nM, 6.25 nM, 3.125 nM). The flow rate was 30 μL/min, the binding and dissociation time were set to 180 s and 800 s or 1200 s, respectively. The chip was regenerated after the last injection of each titration with a glycine solution (pH 2.0) at 30 μL/min for 30 seconds.

Kinetic association rates (kₒₙ) and dissociation rates (k_{off}) were obtained simultaneously by fitting the data globally to a 1:1 Langmuir binding model (Karlsson, R. Roos, H. Fagerstam, L. Petersson, B., 1994. Methods Enzymology 6. 99-110) using Biacore™ 8K Evaluation Software 3.0. Affinity values were deduced from the quotient of the kinetic rate constants (KD=K_{off}/kₒₙ).

As a person of ordinary skill in the art would understand, the same method with appropriate adjustments for parameters (e.g., antibody concentration) was performed for each tested antibody. The results for the tested antibodies are summarized in the table below.

TABLE 2

| Antibody | Protein | kon (1/Ms) | koff (1/s) | KD (M) |
|---|---|---|---|---|
| 12A4 | hIGF1R-His | 2.66E+04 | 5.69E−06 | 2.14E−10 |
| | cIGF1R-His | 1.84E+04 | 1.05E−05 | 5.68E−10 |
| 12D1 | hIGF1R-His | 6.12E+03 | 1.05E−05 | 1.72E−09 |
| | cIGF1R-His | 6.45E+03 | 1.80E−05 | 2.79E−09 |
| 10D1 | hIGF1R-His | 7.75E+03 | 1.26E−05 | 1.63E−09 |
| | cIGF1R-His | 8.36E+03 | 1.75E−05 | 2.10E−09 |
| 10G1 | hIGF1R-His | 4.07E+04 | 1.62E−04 | 3.99E−09 |
| | cIGF1R-His | 2.76E+04 | 2.37E−04 | 8.57E−09 |

The results showed that the anti-IGF1R antibodies 12A4, 12D1, 10D1 and 10G1 had good binding affinities to human IGF1R and monkey IGF1R.

### Example 4. Blocking the Binding of Human IGF-1/2 and IGF1R

Blocking assays were performed to determine whether the anti-IGF1R antibodies can block the binding between IGF1R and its ligand IGF-1 and IGF-2. Specifically, the microwell plates were coated with 100 μl/well of 5 μg/mL hIGF1R-His in pH 9.6 carbonate buffer, overnight at 4° C. Plates were blocked with at 200 μl/well of 1% bovine serum albumin and incubated at 37° C. for 2 hours. Serially diluted anti-IGF1R antibodies (0.005-10 μg/mL in 3-fold serial dilution plus buffer blank) were added to the plates at 100 μL/well. After a 1.5-hour incubation, Biotinylated Human IGF-I Protein (Acro Biosystems, Cat #: IG1-H82F7) or Biotinylated Human IGF-I Protein was replaced with Biotinylated Human IGF-II Protein (Acro Biosystems, Cat #: IG2-H82Q6) were added to the plates at 100 μL/well. After a 1.5-hour incubation at 37° C., HRP Streptavidin (Biolegend, Cat #: 405210) at 1:3000 dilution was added to the plates at 100 μL/well and incubated for 1 hour. Plates were washed 5 times and 100 μL of TMB was added to each well, and incubated for 10 minutes. 100 uL of 1M Phosphoric Acid was then added to each well and ELISA plates were read at 450/570 nm wavelength. A fitting curve was obtained using Log (antibody concentration (M)) as the X-axis and chemiluminescence intensity as the Y-axis. IC50 values were calculated and the results are shown in the table below.

TABLE 3

| | IGF-1 | | IGF-2 | |
|---|---|---|---|---|
| | IC50 | Max inhibition | IC50 | Max inhibition |
| Antibody | (nM) | (%) (10 μg/mL) | (nM) | (%) (10 μg/mL) |
| ISO | — | — | — | — |
| Teprotumumab analog | 1.5 | 45% | 0.9 | 70% |
| 12A4 | 2.0 | 50% | 1.9 | 81% |
| 12D1 | 2.2 | 55% | 1.6 | 88% |
| 10D1 | 1.3 | 47% | 1.7 | 76% |
| 10G1 | 1.6 | 43% | 5.2 | 73% |

All of 12A4, 12D1, 10D1 and 10G1 blocked IGF-1/2 binding to IGF1R with strong inhibition effects.

### Example 5. Anti-IGF1R-Induced Internalization of IGF1R

Anti-IGF1R antibodies (3 μg/mL) and pHAb-AffiniPure Fab Goat Anti-Human IgG Secondary Antibody were added to the NCI-H838 cells (human lung cells, Procell Life Science&Technology Co., Ltd., Cat #: CL-0406). After incubating for 3 hours, the cells were centrifuged and washed with PBS. MFI was detected on a flow cytometer, and EC50 was calculated using serially diluted antibodies.

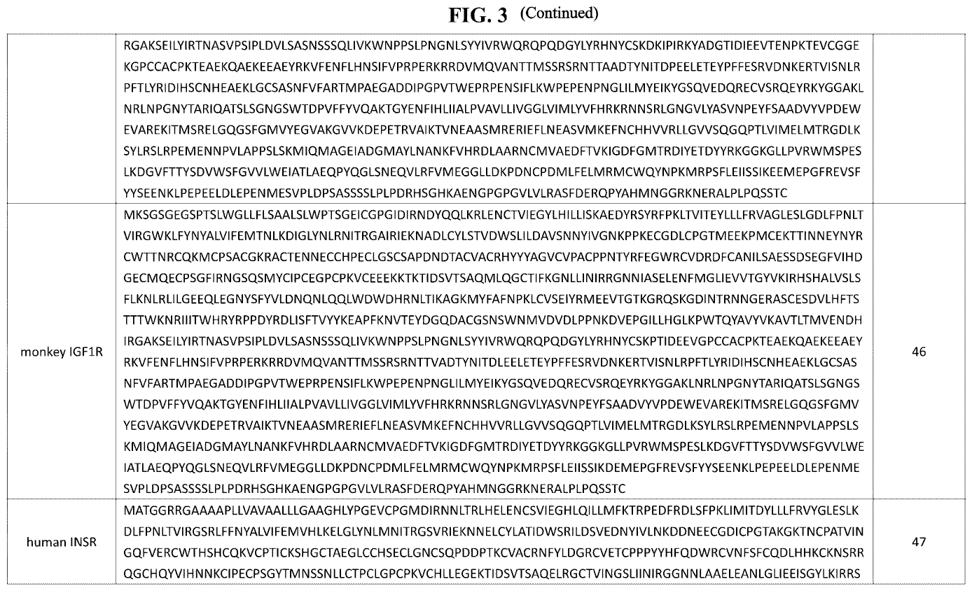

The results are shown in FIG. 4 and Table 4 below, which showed that 12A4, 12D1, 10D1 and 10G1 exhibited comparable endocytosis rates as compared to that of the positive control Teprotumumab analog in NCI-H838 cells.

TABLE 4

| Antibody | EC50 (nM) |
|---|---|
| ISO | — |
| Teprotumumab analog | 0.76 |
| 12A4 | 0.92 |
| 12D1 | 0.66 |
| 10D1 | 0.51 |
| 10G1 | 1.12 |

### Example 6. Epitope Analysis of Anti-IGF1R Antibodies

Epitope binding assays were performed using Biacore™ 8K biosensor to determine whether two anti-IGF1R antibodies target the same or overlapping epitopes. 1×HBS-EP+ buffer (10 mM 4-(2-hydroxyethyl)-1-piperazineethanesulfonic acid (HEPES), 150 mM NaCl, 3 mM ethylenediaminetetraacetic acid (EDTA) and 0.05% P20, pH7.4) diluted from HBS-EP+buffer (10×) was used as the running buffer throughout the experiment. About 50 RU of hIGF1R-His was captured at a flow rate of 10 μL/min, and 200 nM of the tested antibody (Analyte 1) was injected at a flow rate of 30 uL/min to bind the ligand. Another comparison antibody (Analyte 2) was injected under the same conditions to determine whether the binding of different antibodies interfered with each other. The binding time was 180 s for each antibody.

The binding value of each antibody was obtained using Biacore Insight Evaluation (Version: 2.0.15.12933). To quantify the interference of one antibody binding to another, a binding ratio was calculated to compare each pair of antibodies. The binding ratio is defined as the binding value of the second antibody (Analyte 2) divided by the binding value of the first antibody (Analyte 1). The threshold to determine the block or non-block is 0.3. The results were summarized in the table below, which showed that 12A4, 12D1, 10D1, 10G1 and the positive control recognize the same or overlapping epitope.

TABLE 5

| | Analyte 1 | | | | |
|---|---|---|---|---|---|
| | | | | | Teprotumumab |
| Analyte 2 | 12A4 | 12D1 | 10D1 | 10G1 | analog |
| 12A4 | −0.01 | −0.02 | −0.05 | −0.01 | 0.01 |
| 12D1 | 0.00 | −0.01 | −0.04 | 0.00 | 0.01 |
| 10D1 | 0.02 | 0.02 | −0.02 | 0.02 | 0.03 |
| 10G1 | −0.03 | −0.02 | −0.04 | −0.01 | −0.03 |
| Teprotumumab | 0.04 | 0.05 | 0.02 | 0.05 | 0.00 |
| analog | | | | | |

### Example 7. Inhibition of IGF-1-Induced Proliferation

NCI-H838 cells were seeded in 96-well plates at 5,000 cells per well. After a 24-hours incubation, serially diluted anti-IGF1R antibodies (maximum concentration: 10 μg/mL, 5-fold dilutions, 8 gradients) were added to each well and were incubated at 4° C. for 1 hour before the adding of IGF-1 protein. After incubation for 72 hours, the cells were collected and mixed with CellCounting-Lite 2.0 Luminescent Cell Viability Assay (Vazyme, Cat #: DD1101), and tested by microplate reader. IC50 was calculated and the results are shown in the table below.

TABLE 6

| | IC50 | Max inhibition |
|---|---|---|
| Antibody | (nM) | (%) (10 μg/mL) |
| Teprotumumab | 0.400 | 85% |
| analog | | |
| 12A4 | 0.045 | 105% |
| 12D1 | 0.064 | 145% |
| 10D1 | 0.140 | 117% |
| 10G1 | 0.091 | 88% |

The results indicated that 12A4, 12D1 and 10D1 showed almost complete inhibition with max inhibition of more than 100%.

### Example 8. Inhibition of IGF1R Autophosphorylation

The experiment was performed to test whether the anti-IGF1R antibodies can inhibit the phosphorylation of IGF1R. Specifically, CHO-S-hIGF1R cells (2.4×10⁶ cells/mL) were added to a 96-well plate. Anti-IGF1R antibody was added and was incubated at 37° C. for 15 min. Then, IGF-1 protein was added to each well and was incubated for 10 min. After a 5-minutes centrifuge, the supernatant was removed. 50 μL lysis buffer (with Protease and Phosphatase inhibitors) was added to each well and incubated for 30 min on ice. After washed with PBS and centrifuge for 5 min, the sample was transferred to an EILSA plate. Phosphorylated IGF1R levels were measured using Phosphotyrosine IGF1 Receptor ELISA Kit (Abcam, Cat #: ab279824) according to the manufacturer's directions. The mean absorbance for each antibody was determined and the IC50 was calculated.

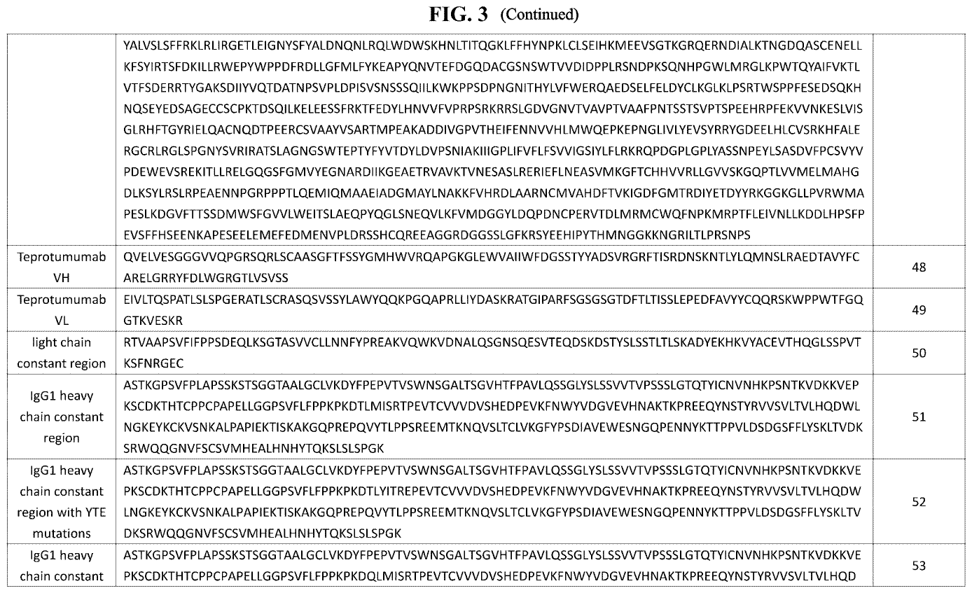

The results are shown in FIGS. 5A-5B and Table 7 below, which demonstrated that 12A4, 12D1, 10D1 and 10G1 exhibited a comparable inhibition effect (e.g. IC50, within 2-fold) of IGF1R phosphorylation as compared to that of the positive control Teprotumumab analog.

TABLE 7

| Antibody | IC50 (nM) |
|---|---|
| Teprotumumab analog | 0.7 |
| 12A4 | 0.9 |
| 12D1 | 1.3 |
| 10D1 | 1.4 |
| 10G1 | 1.2 |

### Example 9. Inhibition of IGF-1-Induced OPN Secretion

Thyroid eye disease (TED) is characterized by swelling and inflammation of orbital and periorbital tissues with widely varying manifestations. Crosstalk between TSHR and IGF1R has been studied in the context of the pathogenesis of TED. The activation of both TSHR and IGF1R in orbital fibroblasts and fibrocytes leads to their differentiation into adipocytes, capable of secreting inflammatory cytokines and hyaluronic acid, and proliferate. In human Osteosarcoma U2OS-TSHR cells (expressing both IGF1R and TSHR), TSHR couples to IGF1R to induce upregulation of genes associated with osteoblast activity (e.g., Osteopontin (OPN)).

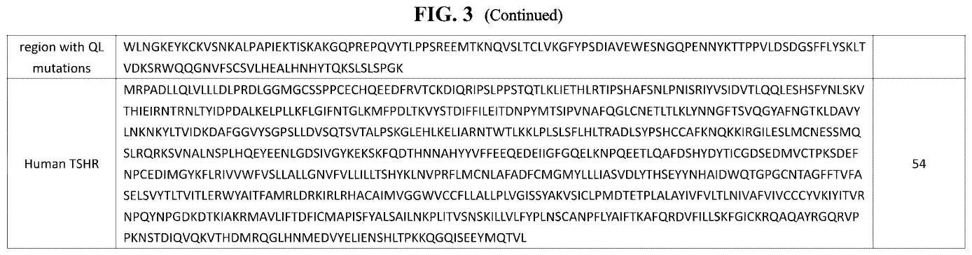

The experiment was performed to test whether the anti-IGF1R antibodies can inhibit IGF-1-induced OPN secretion. Specifically, U2OS-TSHR cells (3× 104 cells/mL, obtained by transfecting U-2OS cells (ATCC, Cat #: HTB-96, endogenously expressing high levels of human IGF-1R) with plasmid expressing human TSHR (SEQ ID NO: 54)) were added to a 24-well plate. Anti-IGF1R antibody or human IgG1 (as ISO control) was added and was incubated at 37° C. for 24 hours. Then, IGF-1 protein was added to each well to induce the secretion of OPN for 24 hours. The cells were collected and centrifuged for 7 days. The secretion levels of OPN were measured by Human Osteopontin (OPN) Quantikine ELISA Kit (R&D systems, Cat #: PDOST00) according to the manufacturer's instructions. The ELISA plates were read at 450 nm wavelength. A fitting curve was obtained using Log (antibody concentration (M)) as the X-axis and human OPN level (ng/mL) as the Y-axis. IC50 values were calculated and the results are shown in FIG. 6 and Table 8 below.

TABLE 8

| Antibody | IC50 (nM) |
|---|---|
| Teprotumumab analog | 0.043 |
| 12A4 | 0.068 |
| 12D1 | 0.096 |
| 10D1 | 0.046 |
| 10G1 | 0.130 |

The results showed that 12A4, 12D1, 10D1 and 10G1 all effectively inhibited IGF-1-stimulated OPN secretion. Specifically, 12A4, 10D1 and 10G1 exhibited a comparable inhibition effect as compared to that of the positive control Teprotumumab analog.

### Example 10. Pharmacokinetics (PK) Analysis

The pharmacokinetic clearance rates of the anti-IGF1R antibodies were determined in B-hFcRn mice (Biocytogen, Cat #: 110001). Specifically, the mice were placed into different groups (3 mice per group), and administered with 3 mg/kg anti-IGF1R antibodies by intravenous injection. Serum samples were collected 4 days before administration and 15 minutes, 1 day, 2 days, 5 days, 7 days, 14 days, 21 days, 28 days, 35 days and 42 days after administration.

The serum levels of human antibodies were determined by sandwich ELISA. Briefly, Goat Anti-Human IgG (H+L) (Jackson ImmunoResearch Inc., Cat #: 109-005-088) was diluted to a final concentration of 2000 ng/mL, added to a 96-well plate (ELISA plate) at 100 μL/well, and then incubated overnight at 4° C. After the incubation, the plate was washed with PBS-T buffer (PBS supplemented with Tween™ 20) 4 times. Antibody-unbound areas were blocked with 2% BSA (bovine serum albumin) for 2 hours at 37° C. Afterwards, the plate was washed with PBS-T buffer 4 times. After washing, 100 μL serum sample was added to each well. The wells were sealed and incubated at 37° C. for 1 hour. After washing the plate using a plate washer, Peroxidase AffiniPure F(ab′)₂ Fragment Goat Anti-Human IgG, Fcγ fragment specific (Jackson ImmunoResearch Inc., Cat #: 109-036-098) was added at 100 μL/well to each well of the plate, and incubated at 37° C. for 1 hour. After washing the plate, tetramethylbenzidine (TMB) solution was added at 100 μL/well to the 96-well plate as the substrate. After incubating at room temperature in the dark, 100 μL stop solution (Beyotime, Cat #: P0215) was added to each well. A microplate reader was used to read the absorbance value of each well at wavelengths of 450 nm and 630 nm. Data analysis software Gen5™ was used to analyze the data. The absorbance value and corresponding concentration of the calibration sample prepared by each test product was used to create a standard curve with four parameters (i.e., T_{1/2}, Cₘₐₓ, AUC_{0-42day}, and CL). The standard curve was used to calculate the antibody concentration of each serum sample. A drug concentration-time curve was created using the calculated sample concentration at each time point. Phoenix™ WinNolin 8.3 was used to calculate the pharmacokinetic parameters. The results are shown in Table 9 below.

TABLE 9

| | T_{1/2} | Cₘₐₓ | AUC_{0-42 day} | CL |
|---|---|---|---|---|
| Group | (day) | (μg/mL) | (day*μg/mL) | (mL/day/kg) |
| Teprotumumab | 9.80 ± 0.25 | 58.60 ± 17.97 | 383.08 ± 33.18 | 7.53 ± 0.64 |
| analog | | | | |
| 12A4-QL | 14.28 ± 1.55 | 56.93 ± 17.55 | 480.82 ± 48.44 | 5.47 ± 0.46 |
| 12A4-YTE | 17.86 ± 1.07 | 85.78 ± 4.18 | 668.88 ± 2.22 | 3.68 ± 0.08 |
| 12D1-QL | 16.95 ± 2.28 | 55.70 ± 3.0 | 365.91±31.66 | 7.09 ± 0.89 |
| 12D1-YTE | 13.86 ± 2.17 | 78.32 ± 3.81 | 509.89 ± 31.54 | 5.16 ± 0.38 |
| 10D1-QL | 15.22 ± 2.45 | 49.78 ± 18.68 | 316.22 ± 78.16 | 7.94 ± 1.78 |
| 10D1-YTE | 15.69 ± 0.61 | 74.53 ± 3.92 | 519.62 ± 4.59 | 4.88 ± 0.10 |
| 10G1-YTE | 16.97 ± 1.16 | 77.52 ± 5.69 | 572.03 ± 22.90 | 4.34 ± 0.12 |

T_{1/2}: Terminal Half Life;
Cₘₐₓ: Max Concentration;
AUC_{0-42 day}: Area under Blood Concentration-time Curve 0-42 day
CL: Clearance

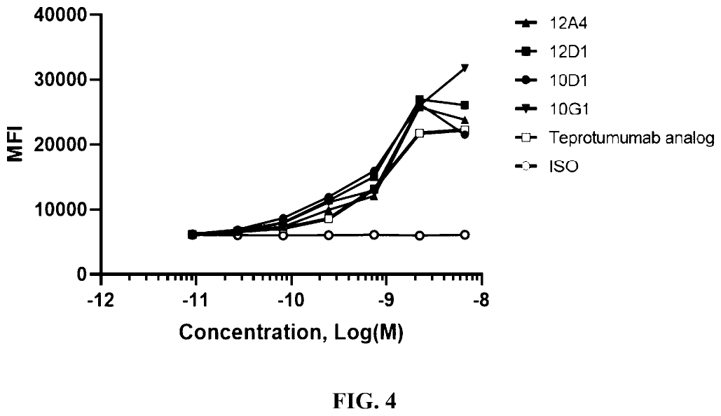

Four days before antibody administration, the antibody concentration was detected as 0 μg/mL (results not shown). As shown in FIG. 7 and Table 9, the results were consistent with typical pharmacokinetic characteristics, showing that after injection of anti-IGF1R antibodies, the concentration of antibodies in the serum of hFcRn mice decreased over time. The tested antibodies displayed longer half-lives and reduced clearance than that of the positive control Teprotumumab analog.

### Example 11. Stability of Anti-IGF1R Antibodies

The experiment was performed to test the stability of the anti-IGF1R antibodies. Anti-IGF1R antibodies were changed to the buffer (20 mM His, 140 mM Arg-HCL, pH5.5) by UF/DF, and their concentration was over 100 mg/ml. Then the antibodies were stored at stressed storage at 40° C. for 4 weeks. Potential molecular variants were measured using SEC-HPLC (TOSOH TSKgel G3000 SWXL, 7.8 mm×30 cm, 5 μm).

The results are shown in Table 10 below. There are no significant decreasing trend in the evaluation of main peak, which demonstrated that 12A4, 12D1, 10G1 and 10D1 have good stability at high concentration.

TABLE 10

| | T0 | | | 4 Weeks | | |
|---|---|---|---|---|---|---|
| | | Main | | | Main | |
| Antibody | HMW | Peak | LMW | HMW | Peak | LMW |
| 12D1 | 3.5% | 96.5% | N.D. | 3.4% | 92.8% | 3.8% |
| 12D1-QL | 3.7% | 96.3% | N.D. | 3.6% | 92.5% | 3.9% |
| 12D1-YTE | 3.4% | 96.6% | N.D. | 4.1% | 91.0% | 5.0% |
| 12A4-QL | 5.5% | 94.5% | N.D. | 4.9% | 89.3% | 5.8% |
| 10D1-YTE | 6.5% | 93.5% | N.D. | 5.7% | 87.8% | 6.6% |
| 10G1-QL | 6.8% | 93.2% | N.D. | 6.1% | 85.4% | 8.5% |

N.D.: Not detected
HMW: High molecular weight
LMW: Low molecular weight

### Example 12. ADCC Activity Assay

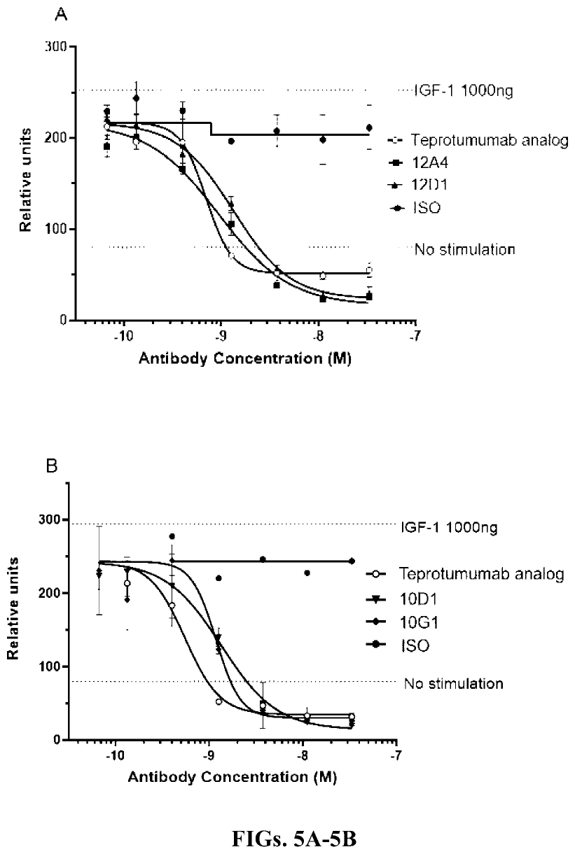

The experiment was performed to test ADCC (antibody-dependent cellular cytotoxicity) activity of anti-IGF1R antibodies. Specifically, effector cells FcR-TANK and target cells Du145 at a E: T ratio of 2:1 were co-incubated with serially diluted anti-IGF1R antibodies for 24 hours, and then the supernatant was collected to test the cell killing. The lactate dehydrogenase (LDH) activity released from the cytosol of damaged cells was measured by non-radioactive colorimetric assay using Cytotoxicity Detection Kit (Roche, Cat #: 04744926001). An antibody with ADCC activity and targeting unrelated target was used as a Reference antibody. The results are shown in FIG. 8, which showed that all tested anti-IGF1R antibodies didn't exhibit ADCC effect.

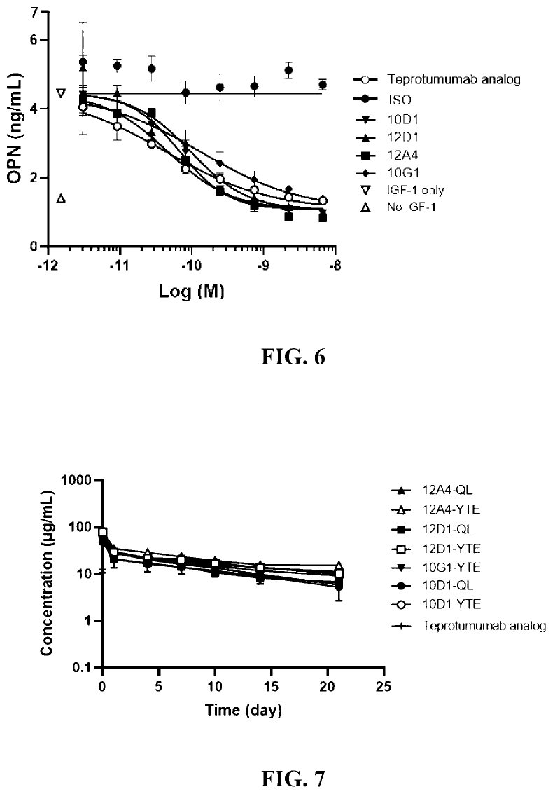

Furthermore, the cytokine INF-y release levels in the same supernatant as the ADCC assay were tested by ELISA. The results are shown in FIG. 9. 12A4-YTE and 12D1-QL treated groups had no change in cytokine levels, while Teprotumumab analog treated group exhibited increased INF-level, indicating that 12A4-YTE and 12D1-QL possessed better safety profile.

### Example 13. HA (Hyaluronan) Release Test

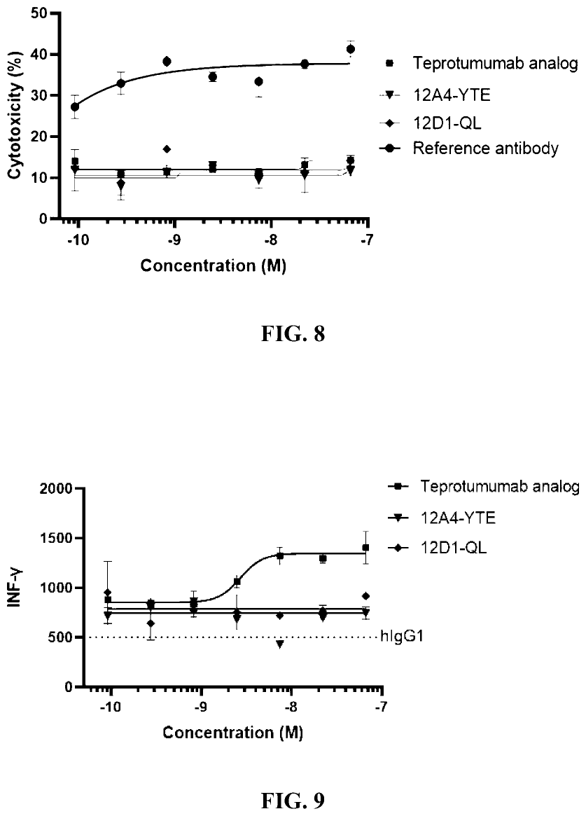

TED is a debilitating disorder characterized by the accumulation of adipocytes and hyaluronan (HA) in orbital space. Production of HA by orbital fibroblasts leads to remarkable increases in tissue volume and to the anterior displacement of the eyes. Fibroblasts isolated from the periorbital space of TED patients were used to test the effect of anti-IGF1R antibodies on HA release. Specifically, fibroblast cells were cultured to confluence. Next, the cells were seeded in a 96-well plate. On the next day, serially diluted anti-IGF1R antibodies (5-fold, the highest concentration 50 μg/mL) were added to each well. Human IgG1 was used as ISO control. Then IGF-1 was added to each well. After the incubation, supernatant from the plate was tested with HA ELISA kit to detect the HA release. FIG. 10 shows that 12A4-YTE and 12D1-QL significantly decreased HA release by in a dose-dependent manner, with 12A4-YTE yielding the largest repression.

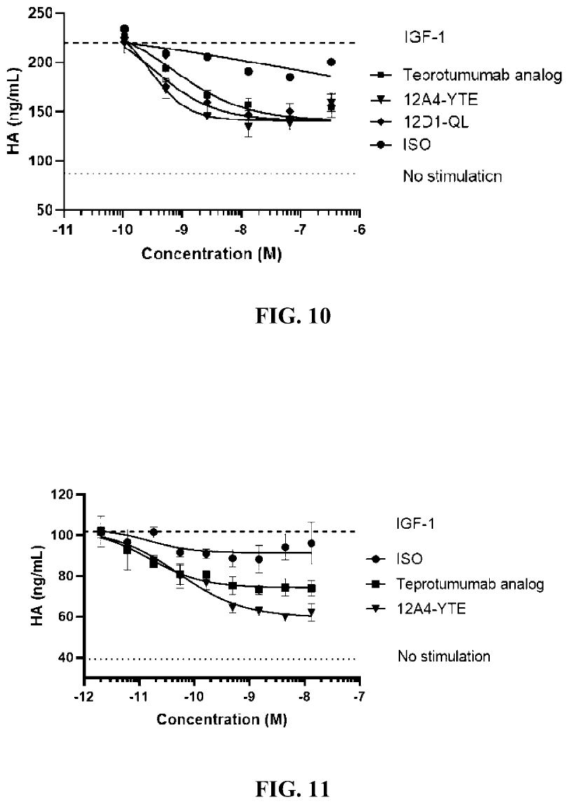

In a similar experiment, fibroblast cells were seeded in a 96-well plate. On the next day, serially diluted anti-IGF1R antibody (3-fold, the highest concentration 2 μg/mL) was added to each well. After incubation with IGF-1, supernatant from the plate was tested with HA ELISA kit to detect the HA release. As shown in FIG. 11, compared with the positive control Teprotumumab analog, 12A4-YTE is more effective in reducing HA release levels, indicating the exciting potential of TED therapies.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An antibody or antigen-binding fragment thereof that binds to insulin-like growth factor 1 receptor (IGF1R), comprising:
a heavy chain variable region (VH) comprising complementarity determining regions (CDRs) 1, 2, and 3, wherein the VH CDR1 region comprises an amino acid sequence that is at least 80% identical to a selected VH CDR1 amino acid sequence, the VH CDR2 region comprises an amino acid sequence that is at least 80% identical to a selected VH CDR2 amino acid sequence, and the VH CDR3 region comprises an amino acid sequence that is at least 80% identical to a selected VH CDR3 amino acid sequence; and
a light chain variable region (VL) comprising CDRs 1, 2, and 3, wherein the VL CDR1 region comprises an amino acid sequence that is at least 80% identical to a selected VL CDR1 amino acid sequence, the VL CDR2 region comprises an amino acid sequence that is at least 80% identical to a selected VL CDR2 amino acid sequence, and the VL CDR3 region comprises an amino acid sequence that is at least 80% identical to a selected VL CDR3 amino acid sequence,
wherein the selected VH CDRs 1, 2, and 3 amino acid sequences and the selected VL CDRs, 1, 2, and 3 amino acid sequences are one of the following:
(1) the selected VH CDRs 1, 2, 3 amino acid sequences are set forth in SEQ ID NOs: 1, 2, 3, respectively, and the selected VL CDRs 1, 2, 3 amino acid sequences are set forth in SEQ ID NOs: 4, 5, 6, respectively;
(2) the selected VH CDRs 1, 2, 3 amino acid sequences are set forth in SEQ ID NOs: 25, 26, 27, respectively, and the selected VL CDRs 1, 2, 3 amino acid sequences are set forth in SEQ ID NOs: 4, 5, 6, respectively;
(3) the selected VH CDRs 1, 2, 3 amino acid sequences are set forth in SEQ ID NOs: 7, 8, 9, respectively, and the selected VL CDRs 1, 2, 3 amino acid sequences are set forth in SEQ ID NOs: 10, 11, 12, respectively;
(4) the selected VH CDRs 1, 2, 3 amino acid sequences are set forth in SEQ ID NOs: 28, 29, 30, respectively, and the selected VL CDRs 1, 2, 3 amino acid sequences are set forth in SEQ ID NOs: 10, 11, 12, respectively;
(5) the selected VH CDRs 1, 2, 3 amino acid sequences are set forth in SEQ ID NOs: 13, 14, 15, respectively, and the selected VL CDRs 1, 2, 3 amino acid sequences are set forth in SEQ ID NOs: 16, 17, 18, respectively;
(6) the selected VH CDRs 1, 2, 3 amino acid sequences are set forth in SEQ ID NOs: 31, 32, 33, respectively, and the selected VL CDRs 1, 2, 3 amino acid sequences are set forth in SEQ ID NOs: 16, 17, 18, respectively;
(7) the selected VH CDRs 1, 2, 3 amino acid sequences are set forth in SEQ ID NOs: 19, 20, 21, respectively, and the selected VL CDRs 1, 2, 3 amino acid sequences are set forth in SEQ ID NOs: 22, 23, 24, respectively;
(8) the selected VH CDRs 1, 2, 3 amino acid sequences are set forth in SEQ ID NOs: 34, 35, 36, respectively, and the selected VL CDRs 1, 2, 3 amino acid sequences are set forth in SEQ ID NOs: 22, 23, 24, respectively.

2. The antibody or antigen-binding fragment thereof of claim 1, wherein the VH comprises CDRs 1, 2, 3 with the amino acid sequences set forth in SEQ ID NOs: 1, 2, and 3, respectively, and the VL comprises CDRs 1, 2, 3 with the amino acid sequences set forth in SEQ ID NOs: 4, 5, and 6, respectively, according to Kabat definition.

3. The antibody or antigen-binding fragment thereof of claim 1, wherein the VH comprises CDRs 1, 2, 3 with the amino acid sequences set forth in SEQ ID NOs: 7, 8, and 9, respectively, and the VL comprises CDRs 1, 2, 3 with the amino acid sequences set forth in SEQ ID NOs: 10, 11, and 12, respectively, according to Kabat definition.

4. The antibody or antigen-binding fragment thereof of claim 1, wherein the VH comprises CDRs 1, 2, 3 with the amino acid sequences set forth in SEQ ID NOs: 13, 14, and 15, respectively, and the VL comprises CDRs 1, 2, 3 with the amino acid sequences set forth in SEQ ID NOs: 16, 17 and 18, respectively, according to Kabat definition.

5. The antibody or antigen-binding fragment thereof of claim 1, wherein the VH comprises CDRs 1, 2, 3 with the amino acid sequences set forth in SEQ ID NOs: 19, 20, and 21, respectively, and the VL comprises CDRs 1, 2, 3 with the amino acid sequences set forth in SEQ ID NOs: 22, 23, and 24, respectively, according to Kabat definition.

6. The antibody or antigen-binding fragment thereof of claim 1, wherein the VH comprises CDRs 1, 2, 3 with the amino acid sequences set forth in SEQ ID NOs: 25, 26, and 27, respectively, and the VL comprises CDRs 1, 2, 3 with the amino acid sequences set forth in SEQ ID NOs: 4, 5, and 6, respectively, according to Chothia definition.

7. The antibody or antigen-binding fragment thereof of claim 1, wherein the VH comprises CDRs 1, 2, 3 with the amino acid sequences set forth in SEQ ID NOs: 28, 29, and 30, respectively, and the VL comprises CDRs 1, 2, 3 with the amino acid sequences set forth in SEQ ID NOs: 10, 11, and 12, respectively, according to Chothia definition.

8. The antibody or antigen-binding fragment thereof of claim 1, wherein the VH comprises CDRs 1, 2, 3 with the amino acid sequences set forth in SEQ ID NOs: 31, 32, and 33, respectively, and the VL comprises CDRs 1, 2, 3 with the amino acid sequences set forth in SEQ ID NOs: 16, 17 and 18, respectively, according to Chothia definition.

9. The antibody or antigen-binding fragment thereof of claim 1, wherein the VH comprises CDRs 1, 2, 3 with the amino acid sequences set forth in SEQ ID NOs: 34, 35, and 36, respectively, and the VL comprises CDRs 1, 2, 3 with the amino acid sequences set forth in SEQ ID NOs: 22, 23, and 24, respectively, according to Chothia definition.

10. The antibody or antigen-binding fragment thereof of any one of claims 1-9, wherein the antibody or antigen-binding fragment specifically binds to human IGF1R or monkey IGF1R.

11. The antibody or antigen-binding fragment thereof of any one of claims 1-10, wherein the antibody or antigen-binding fragment is a human or humanized antibody or antigen-binding fragment thereof (e.g., a human IgG1 or IgG4 antibody).

12. The antibody or antigen-binding fragment thereof of any one of claims 1-11, wherein the antibody or antigen-binding fragment is a single-chain variable fragment (scFv) or a multi-specific antibody (e.g., a bispecific antibody).

13. A nucleic acid comprising a polynucleotide encoding a polypeptide comprising:
(1) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 1, 2, and 3, respectively, and wherein the VH, when paired with a light chain variable region (VL) comprising the amino acid sequence set forth in SEQ ID NO: 38 binds to IGF1R;
(2) an immunoglobulin light chain or a fragment thereof comprising a VL comprising CDRs 1, 2, and 3 comprising the amino acid sequences set forth in SEQ ID NOs: 4, 5, and 6, respectively, and wherein the VL, when paired with a VH comprising the amino acid sequence set forth in SEQ ID NO: 37 binds to IGF1R;
(3) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 25, 26, and 27, respectively, and wherein the VH, when paired with a light chain variable region (VL) comprising the amino acid sequence set forth in SEQ ID NO: 38 binds to IGF1R;
(4) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 7, 8, and 9, respectively, and wherein the VH, when paired with a light chain variable region (VL) comprising the amino acid sequence set forth in SEQ ID NO: 40 binds to IGF1R;
(5) an immunoglobulin light chain or a fragment thereof comprising a VL comprising CDRs 1, 2, and 3 comprising the amino acid sequences set forth in SEQ ID NOs: 10, 11, and 12, respectively, and wherein the VL, when paired with a VH comprising the amino acid sequence set forth in SEQ ID NO: 39 binds to IGF1R;
(6) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 28, 29, and 30, respectively, and wherein the VH, when paired with a light chain variable region (VL) comprising the amino acid sequence set forth in SEQ ID NO: 40 binds to IGF1R;
(7) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 13, 14, and 15, respectively, and wherein the VH, when paired with a light chain variable region (VL) comprising the amino acid sequence set forth in SEQ ID NO: 42 binds to IGF1R;
(8) an immunoglobulin light chain or a fragment thereof comprising a VL comprising CDRs 1, 2, and 3 comprising the amino acid sequences set forth in SEQ ID NOs: 16, 17, and 18, respectively, and wherein the VL, when paired with a VH comprising the amino acid sequence set forth in SEQ ID NO: 41 binds to IGF1R;
(9) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 31, 32, and 33, respectively, and wherein the VH, when paired with a light chain variable region (VL) comprising the amino acid sequence set forth in SEQ ID NO: 42 binds to IGF1R;
(10) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 19, 20 and 21, respectively, and wherein the VH, when paired with a light chain variable region (VL) comprising the amino acid sequence set forth in SEQ ID NO: 44 binds to IGF1R;
(11) an immunoglobulin light chain or a fragment thereof comprising a VL comprising CDRs 1, 2, and 3 comprising the amino acid sequences set forth in SEQ ID NOs: 22, 23, and 24, respectively, and wherein the VL, when paired with a VH comprising the amino acid sequence set forth in SEQ ID NO: 43 binds to IGF1R; or
(12) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 34, 35, and 36, respectively, and wherein the VH, when paired with a light chain variable region (VL) comprising the amino acid sequence set forth in SEQ ID NO: 44 binds to IGF1R.

14. The nucleic acid of claim 13, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 1, 2, and 3, respectively, or SEQ ID NOs: 25, 26, and 27, respectively.

15. The nucleic acid of claim 13, wherein the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 4, 5, and 6, respectively.

16. The nucleic acid of claim 13, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 7, 8, and 9, respectively, or SEQ ID NOs: 28, 29, and 30, respectively.

17. The nucleic acid of claim 13, wherein the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 10, 11, and 12, respectively.

18. The nucleic acid of claim 13, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 13, 14, and 15, respectively, or SEQ ID NOs: 31, 32, and 33, respectively.

19. The nucleic acid of claim 13, wherein the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 16, 17, and 18, respectively.

20. The nucleic acid of claim 13, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 19, 20, and 21, respectively, or SEQ ID NOs: 34, 35, and 36, respectively.

21. The nucleic acid of claim 13, wherein the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 22, 23, and 24, respectively.

22. The nucleic acid of any one of claims 13-21, wherein the VH when paired with a VL specifically binds to human IGF1R or monkey IGF1R, or the VL when paired with a VH specifically binds to human IGF1R or monkey IGF1R.

23. The nucleic acid of any one of claims 13-22, wherein the immunoglobulin heavy chain or the fragment thereof is a human or humanized immunoglobulin heavy chain or a fragment thereof (e.g., a human IgG1 heavy chain or a fragment thereof), and the immunoglobulin light chain or the fragment thereof is a human or humanized immunoglobulin light chain or a fragment thereof.

24. The nucleic acid of any one of claims 13-23, wherein the nucleic acid encodes a single-chain variable fragment (scFv), a multi-specific antibody (e.g., a bispecific antibody) or a chimeric antigen receptor (CAR).

25. The nucleic acid of any one of claims 13-24, wherein the nucleic acid is cDNA.

26. A vector comprising one or more of the nucleic acids of any one of claims 13-25.

27. A vector comprising two of the nucleic acids of any one of claims 13-25, wherein the vector encodes the VH region and the VL region that together bind to IGF1R.

28. A pair of vectors, wherein each vector comprises one of the nucleic acids of any one of claims 13-25, wherein together the pair of vectors encodes the VH region and the VL region that together bind to IGF1R.

29. A cell comprising the vector of claim 26 or 27, or the pair of vectors of claim 28.

30. The cell of claim 29, wherein the cell is a CHO cell.

31. A cell comprising one or more of the nucleic acids of any one of claims 13-25.

32. A cell comprising two of the nucleic acids of any one of claims 13-25.

33. The cell of claim 32, wherein the two nucleic acids together encode the VH region and the VL region that together bind to IGF1R.

34. A method of producing an antibody or an antigen-binding fragment thereof, the method comprising
(a) culturing the cell of any one of claims 29-33 under conditions sufficient for the cell to produce the antibody or the antigen-binding fragment; and
(b) collecting the antibody or the antigen-binding fragment produced by the cell.

35. An antibody or antigen-binding fragment thereof that binds to IGF1R comprising
a heavy chain variable region (VH) comprising an amino acid sequence that is at least 80% identical to a selected VH sequence, and a light chain variable region (VL) comprising an amino acid sequence that is at least 80% identical to a selected VL sequence, wherein the selected VH sequence and the selected VL sequence are one of the following:
(1) the selected VH sequence is SEQ ID NO: 37, and the selected VL sequence is SEQ ID NO: 38;
(2) the selected VH sequence is SEQ ID NO: 39, and the selected VL sequence is SEQ ID NO: 40;
(3) the selected VH sequence is SEQ ID NO: 41, and the selected VL sequence is SEQ ID NO: 42; and
(4) the selected VH sequence is SEQ ID NO: 43, and the selected VL sequence is SEQ ID NO: 44.

36. The antibody or antigen-binding fragment thereof of claim 35, wherein the VH comprises the sequence of SEQ ID NO: 37 and the VL comprises the sequence of SEQ ID NO: 38.

37. The antibody or antigen-binding fragment thereof of claim 35, wherein the VH comprises the sequence of SEQ ID NO: 39 and the VL comprises the sequence of SEQ ID NO: 40.

38. The antibody or antigen-binding fragment thereof of claim 35, wherein the VH comprises the sequence of SEQ ID NO: 41 and the VL comprises the sequence of SEQ ID NO: 42.

39. The antibody or antigen-binding fragment thereof of claim 35, wherein the VH comprises the sequence of SEQ ID NO: 43 and the VL comprises the sequence of SEQ ID NO: 44.

40. The antibody or antigen-binding fragment thereof of any one of claims 35-39, wherein the antibody or antigen-binding fragment specifically binds to human IGF1R or monkey IGF1R.

41. The antibody or antigen-binding fragment thereof of any one of claims 35-40, wherein the antibody or antigen-binding fragment is a human or humanized antibody or antigen-binding fragment thereof.

42. The antibody or antigen-binding fragment thereof of any one of claims 35-41, wherein the antibody or antigen-binding fragment is a single-chain variable fragment (scFv) or a multi-specific antibody (e.g., a bispecific antibody).

43. An antibody or antigen-binding fragment thereof that cross-competes with the antibody or antigen-binding fragment thereof of any one of claims 1-12 and 35-42.

44. An antibody or antigen-binding fragment thereof that binds to IGF1R comprising
a heavy chain variable region (VH) comprising VH CDR1, VH CDR2, and VH CDR3 that are identical to VH CDR1, VH CDR2, and VH CDR3 of a selected VH sequence; and
a light chain variable region (VL) comprising VL CDR1, VL CDR2, and VL CDR3 that are identical to VL CDR1, VL CDR2, and VL CDR3 of a selected VL sequence, wherein the selected VH sequence and the selected VL sequence are one of the following:
(1) the selected VH sequence is SEQ ID NO: 37, and the selected VL sequence is SEQ ID NO: 38;
(2) the selected VH sequence is SEQ ID NO: 39, and the selected VL sequence is SEQ ID NO: 40;
(3) the selected VH sequence is SEQ ID NO: 41, and the selected VL sequence is SEQ ID NO: 42; and
(4) the selected VH sequence is SEQ ID NO: 43, and the selected VL sequence is SEQ ID NO: 44.

45. An antibody-drug conjugate comprising the antibody or antigen-binding fragment thereof of any one of claims 1-12 and 35-44 covalently bound to a therapeutic agent.

46. The antibody drug conjugate of claim 45, wherein the therapeutic agent is a cytotoxic or cytostatic agent.

47. A method of treating a subject having cancer, the method comprising administering a therapeutically effective amount of a composition comprising the antibody or antigen-binding fragment thereof of any one of claims 1-12 and 35-44, or the antibody-drug conjugate of claim 45 or 46, to the subject.

48. The method of claim 47, wherein the subject has breast cancer, prostate cancer, or lung cancer.

49. The method of claim 47 or 48, further comprising administering a therapeutically effective amount of an anti-CD3 antibody, an anti-PD1 antibody, an anti-PDL1 antibody, an anti-HER2 antibody, and/or an anti-EGFR antibody, to the subject.

50. A method of decreasing the rate of tumor growth, the method comprising contacting a tumor cell with an effective amount of a composition comprising an antibody or antigen-binding fragment thereof of any one of claims 1-12 and 35-44, or the antibody-drug conjugate of claim 45 or 46.

51. A method of killing a tumor cell, the method comprising contacting a tumor cell with an effective amount of a composition comprising the antibody or antigen-binding fragment thereof of any one of claims 1-12 and 35-44, or the antibody-drug conjugate of claim 45 or 46.

52. A method of treating a subject having an eye disease or disorder, the method comprising administering a therapeutically effective amount of a composition comprising the antibody or antigen-binding fragment thereof of any one of claims 1-12 and 35-44, or the antibody-drug conjugate of claim 45 or 46, to the subject.

53. The method of claim 52, wherein the subject has thyroid eye disease (TED).

54. A method of treating a subject having a bone disease or disorder, the method comprising administering a therapeutically effective amount of a composition comprising the antibody or antigen-binding fragment thereof of any one of claims 1-12 and 35-44, or the antibody-drug conjugate of claim 45 or 46, to the subject.

55. A pharmaceutical composition comprising the antibody or antigen-binding fragment thereof of any one of claims 1-12 and 35-44, and a pharmaceutically acceptable carrier.

56. A pharmaceutical composition comprising the antibody drug conjugate of claim 45 or 46, and a pharmaceutically acceptable carrier.

57. A chimeric antigen receptor (CAR) comprising the antibody or antigen-binding fragment thereof of any one of claims 1-12 and 35-44.


SEQUENCE LISTING (54 sequences)

SEQ ID NO: 1 - protein, 5 aa, synthetic construct
DYGMS

SEQ ID NO: 2 - protein, 17 aa, synthetic construct
EIDWTGGSKLYADSVKG

SEQ ID NO: 3 - protein, 16 aa, synthetic construct
DRGAVSPKYYYYGMDV

SEQ ID NO: 4 - protein, 11 aa, synthetic construct
RASQGIRNDLG

SEQ ID NO: 5 - protein, 7 aa, synthetic construct
AASRLQS

SEQ ID NO: 6 - protein, 9 aa, synthetic construct
LQHNSYPYT

SEQ ID NO: 7 - protein, 5 aa, synthetic construct
DYGMS

SEQ ID NO: 8 - protein, 17 aa, synthetic construct
GINWNGGSTDYADSVKG

SEQ ID NO: 9 - protein, 16 aa, synthetic construct
DRGAVTRRYYYYGMDV

SEQ ID NO: 10 - protein, 11 aa, synthetic construct
RASQDIRNDLG

SEQ ID NO: 11 - protein, 7 aa, synthetic construct
AASRLQS

SEQ ID NO: 12 - protein, 9 aa, synthetic construct
LQHNSYPYT

SEQ ID NO: 13 - protein, 5 aa, synthetic construct
DYGMS

SEQ ID NO: 14 - protein, 17 aa, synthetic construct
GINWNGGSTDYADSVKG

SEQ ID NO: 15 - protein, 16 aa, synthetic construct
DRGAVSRRYYYYGMDV

SEQ ID NO: 16 - protein, 11 aa, synthetic construct
RASQDIRNDLG

SEQ ID NO: 17 - protein, 7 aa, synthetic construct
AASRLQS

SEQ ID NO: 18 - protein, 9 aa, synthetic construct
LQHNSYPYT

SEQ ID NO: 19 - protein, 5 aa, synthetic construct
DYYMS

SEQ ID NO: 20 - protein, 17 aa, synthetic construct
YISNSGSFTNYADSVKG

SEQ ID NO: 21 - protein, 16 aa, synthetic construct
DQAPVTSKYYYYGMDV

SEQ ID NO: 22 - protein, 11 aa, synthetic construct
RASQGIRNDFA

SEQ ID NO: 23 - protein, 7 aa, synthetic construct
AASRLQR

SEQ ID NO: 24 - protein, 9 aa, synthetic construct
LQHNSYPYT

SEQ ID NO: 25 - protein, 7 aa, synthetic construct
GFTFADY

SEQ ID NO: 26 - protein, 6 aa, synthetic construct
DWTGGS

SEQ ID NO: 27 - protein, 16 aa, synthetic construct
DRGAVSPKYYYYGMDV

SEQ ID NO: 28 - protein, 7 aa, synthetic construct
GFTFDDY

SEQ ID NO: 29 - protein, 6 aa, synthetic construct
NWNGGS

SEQ ID NO: 30 - protein, 16 aa, synthetic construct
DRGAVTRRYYYYGMDV

SEQ ID NO: 31 - protein, 7 aa, synthetic construct
GFTFADY

SEQ ID NO: 32 - protein, 6 aa, synthetic construct
NWNGGS

SEQ ID NO: 33 - protein, 16 aa, synthetic construct
DRGAVSRRYYYYGMDV

SEQ ID NO: 34 - protein, 7 aa, synthetic construct
EFTFSDY

SEQ ID NO: 35 - protein, 6 aa, synthetic construct
SNSGSF

SEQ ID NO: 36 - protein, 16 aa, synthetic construct
DQAPVTSKYYYYGMDV

SEQ ID NO: 37 - protein, 125 aa, synthetic construct
EVQLVQSGGGEIRPGGSLRLSCAASGFTFADYGMSWVRQAPGEGLEWVSEIDWTGGSKLYADSVKGRFTISRDNAKNSLFLQMNSLRAEDTALYYCVRDRGAVSPKYYYYGMDVWGQGTTVTVSS

SEQ ID NO: 38 - protein, 107 aa, synthetic construct
EIVLTQSPSSLSASVGDRVTITCRASQGIRNDLGWYQQKPGKAPKRLIYAASRLQSGVPSRFSGSGSGTDFTLTISSLQPEDFATYYCLQHNSYPYTFGQGTKLEIK

SEQ ID NO: 39 - protein, 125 aa, synthetic construct
QVQLVESGGGVVRPGGSLRLSCAASGFTFDDYGMSWVRQVPGKGLEWVSGINWNGGSTDYADSVKGRFTISREKAKNSLYLQMNSLRAEDTALYYCVRDRGAVTRRYYYYGMDVWGQGTTVTVSS

SEQ ID NO: 40 - protein, 107 aa, synthetic construct
DIQMTQSPSSLSASVGDRVTITCRASQDIRNDLGWYQLKPGKAPKRLIYAASRLQSGVPSRFSGSGSGTEFTLTISSLQPEDFATYYCLQHNSYPYTFGQGTKLEIK

SEQ ID NO: 41 - protein, 125 aa, synthetic construct
QVQLQQSGGGVVRPGGSLRLSCAASGFTFADYGMSWVRQVPGKGLEWVSGINWNGGSTDYADSVKGRFTISRDNAKHSLYLQMNSLRAEDTAFYYCARDRGAVSRRYYYYGMDVWGLGTTVTVSS

SEQ ID NO: 42 - protein, 107 aa, synthetic construct
AIQLTQSPSSLSASVGDRVTITCRASQDIRNDLGWYQLKPGKAPKRLIYAASRLQSGVPSGFSGSGSGTEFTLTISSLQPEDFATYYCLQHNSYPYTFGQGTKLEIK

SEQ ID NO: 43 - protein, 125 aa, synthetic construct
QVQLVESGGGLVKPGGSLRLSCAASEFTFSDYYMSWIRQSPGKGLEWVSYISNSGSFTNYADSVKGRFTISRDNAKNSLYLQMNSLRAEDSAIYYCVRDQAPVTSKYYYYGMDVWGQGTTVTVSS

SEQ ID NO: 44 - protein, 107 aa, synthetic construct
EIVLTQSPSSLSASVGDRVTITCRASQGIRNDFAWYQQKPGKAPKRLIYAASRLQRGVPSRFSGSGSGTEFTLTISSLQPEDFATYYCLQHNSYPYTFGQGTKLEIK

SEQ ID NO: 45 - protein, 1367 aa, Homo sapiens
MKSGSGGGSPTSLWGLLFLSAALSLWPTSGEICGPGIDIRNDYQQLKRLENCTVIEGYLHILLISKAEDYRSYRFPKLTVITEYLLLFRVAGLESLGDLFPNLTVIRGWKLFYNYALVIFEMTNLKDIGLYNLRNITRGAIRIEKNADLCYLSTVDWSLILDAVSNNYIVGNKPPKECGDLCPGTMEEKPMCEKTTINNEYNYRCWTTNRCQKMCPSTCGKRACTENNECCHPECLGSCSAPDNDTACVACRHYYYAGVCVPACPPNTYRFEGWRCVDRDFCANILSAESSDSEGFVIHDGECMQECPSGFIRNGSQSMYCIPCEGPCPKVCEEEKKTKTIDSVTSAQMLQGCTIFKGNLLINIRRGNNIASELENFMGLIEVVTGYVKIRHSHALVSLSFLKNLRLILGEEQLEGNYSFYVLDNQNLQQLWDWDHRNLTIKAGKMYFAFNPKLCVSEIYRMEEVTGTKGRQSKGDINTRNNGERASCESDVLHFTSTTTSKNRIIITWHRYRPPDYRDLISFTVYYKEAPFKNVTEYDGQDACGSNSWNMVDVDLPPNKDVEPGILLHGLKPWTQYAVYVKAVTLTMVENDHIRGAKSEILYIRTNASVPSIPLDVLSASNSSSQLIVKWNPPSLPNGNLSYYIVRWQRQPQDGYLYRHNYCSKDKIPIRKYADGTIDIEEVTENPKTEVCGGEKGPCCACPKTEAEKQAEKEEAEYRKVFENFLHNSIFVPRPERKRRDVMQVANTTMSSRSRNTTAADTYNITDPEELETEYPFFESRVDNKERTVISNLRPFTLYRIDIHSCNHEAEKLGCSASNFVFARTMPAEGADDIPGPVTWEPRPENSIFLKWPEPENPNGLILMYEIKYGSQVEDQRECVSRQEYRKYGGAKLNRLNPGNYTARIQATSLSGNGSWTDPVFFYVQAKTGYENFIHLIIALPVAVLLIVGGLVIMLYVFHRKRNNSRLGNGVLYASVNPEYFSAADVYVPDEWEVAREKITMSRELGQGSFGMVYEGVAKGVVKDEPETRVAIKTVNEAASMRERIEFLNEASVMKEFNCHHVVRLLGVVSQGQPTLVIMELMTRGDLKSYLRSLRPEMENNPVLAPPSLSKMIQMAGEIADGMAYLNANKFVHRDLAARNCMVAEDFTVKIGDFGMTRDIYETDYYRKGGKGLLPVRWMSPESLKDGVFTTYSDVWSFGVVLWEIATLAEQPYQGLSNEQVLRFVMEGGLLDKPDNCPDMLFELMRMCWQYNPKMRPSFLEIISSIKEEMEPGFREVSFYYSEENKLPEPEELDLEPENMESVPLDPSASSSSLPLPDRHSGHKAENGPGPGVLVLRASFDERQPYAHMNGGRKNERALPLPQSSTC

SEQ ID NO: 46 - protein, 1343 aa, Macaca fascicularis
MKSGSGEGSPTSLWGLLFLSAALSLWPTSGEICGPGIDIRNDYQQLKRLENCTVIEGYLHILLISKAEDYRSYRFPKLTVITEYLLLFRVAGLESLGDLFPNLTVIRGWKLFYNYALVIFEMTNLKDIGLYNLRNITRGAIRIEKNADLCYLSTVDWSLILDAVSNNYIVGNKPPKECGDLCPGTMEEKPMCEKTTINNEYNYRCWTTNRCQKMCPSACGKRACTENNECCHPECLGSCSAPDNDTACVACRHYYYAGVCVPACPPNTYRFEGWRCVDRDFCANILSAESSDSEGFVIHDGECMQECPSGFIRNGSQSMYCIPCEGPCPKVCEEEKKTKTIDSVTSAQMLQGCTIFKGNLLINIRRGNNIASELENFMGLIEVVTGYVKIRHSHALVSLSFLKNLRLILGEEQLEGNYSFYVLDNQNLQQLWDWDHRNLTIKAGKMYFAFNPKLCVSEIYRMEEVTGTKGRQSKGDINTRNNGERASCESDVLHFTSTTTWKNRIIITWHRYRPPDYRDLISFTVYYKEAPFKNVTEYDGQDACGSNSWNMVDVDLPPNKDVEPGILLHGLKPWTQYAVYVKAVTLTMVENDHIRGAKSEILYIRTNASVPSIPLDVLSASNSSSQLIVKWNPPSLPNGNLSYYIVRWQRQPQDGYLYRHNYCSKPTIDEEVGPCCACPKTEAEKQAEKEEAEYRKVFENFLHNSIFVPRPERKRRDVMQVANTTMSSRSRNTTVADTYNITDLEELETEYPFFESRVDNKERTVISNLRPFTLYRIDIHSCNHEAEKLGCSASNFVFARTMPAEGADDIPGPVTWEPRPENSIFLKWPEPENPNGLILMYEIKYGSQVEDQRECVSRQEYRKYGGAKLNRLNPGNYTARIQATSLSGNGSWTDPVFFYVQAKTGYENFIHLIIALPVAVLLIVGGLVIMLYVFHRKRNNSRLGNGVLYASVNPEYFSAADVYVPDEWEVAREKITMSRELGQGSFGMVYEGVAKGVVKDEPETRVAIKTVNEAASMRERIEFLNEASVMKEFNCHHVVRLLGVVSQGQPTLVIMELMTRGDLKSYLRSLRPEMENNPVLAPPSLSKMIQMAGEIADGMAYLNANKFVHRDLAARNCMVAEDFTVKIGDFGMTRDIYETDYYRKGGKGLLPVRWMSPESLKDGVFTTYSDVWSFGVVLWEIATLAEQPYQGLSNEQVLRFVMEGGLLDKPDNCPDMLFELMRMCWQYNPKMRPSFLEIISSIKDEMEPGFREVSFYYSEENKLPEPEELDLEPENMESVPLDPSASSSSLPLPDRHSGHKAENGPGPGVLVLRASFDERQPYAHMNGGRKNERALPLPQSSTC

SEQ ID NO: 47 - protein, 1370 aa, Homo sapiens
MATGGRRGAAAAPLLVAVAALLLGAAGHLYPGEVCPGMDIRNNLTRLHELENCSVIEGHLQILLMFKTRPEDFRDLSFPKLIMITDYLLLFRVYGLESLKDLFPNLTVIRGSRLFFNYALVIFEMVHLKELGLYNLMNITRGSVRIEKNNELCYLATIDWSRILDSVEDNYIVLNKDDNEECGDICPGTAKGKTNCPATVINGQFVERCWTHSHCQKVCPTICKSHGCTAEGLCCHSECLGNCSQPDDPTKCVACRNFYLDGRCVETCPPPYYHFQDWRCVNFSFCQDLHHKCKNSRRQGCHQYVIHNNKCIPECPSGYTMNSSNLLCTPCLGPCPKVCHLLEGEKTIDSVTSAQELRGCTVINGSLIINIRGGNNLAAELEANLGLIEEISGYLKIRRSYALVSLSFFRKLRLIRGETLEIGNYSFYALDNQNLRQLWDWSKHNLTITQGKLFFHYNPKLCLSEIHKMEEVSGTKGRQERNDIALKTNGDQASCENELLKFSYIRTSFDKILLRWEPYWPPDFRDLLGFMLFYKEAPYQNVTEFDGQDACGSNSWTVVDIDPPLRSNDPKSQNHPGWLMRGLKPWTQYAIFVKTLVTFSDERRTYGAKSDIIYVQTDATNPSVPLDPISVSNSSSQIILKWKPPSDPNGNITHYLVFWERQAEDSELFELDYCLKGLKLPSRTWSPPFESEDSQKHNQSEYEDSAGECCSCPKTDSQILKELEESSFRKTFEDYLHNVVFVPRPSRKRRSLGDVGNVTVAVPTVAAFPNTSSTSVPTSPEEHRPFEKVVNKESLVISGLRHFTGYRIELQACNQDTPEERCSVAAYVSARTMPEAKADDIVGPVTHEIFENNVVHLMWQEPKEPNGLIVLYEVSYRRYGDEELHLCVSRKHFALERGCRLRGLSPGNYSVRIRATSLAGNGSWTEPTYFYVTDYLDVPSNIAKIIIGPLIFVFLFSVVIGSIYLFLRKRQPDGPLGPLYASSNPEYLSASDVFPCSVYVPDEWEVSREKITLLRELGQGSFGMVYEGNARDIIKGEAETRVAVKTVNESASLRERIEFLNEASVMKGFTCHHVVRLLGVVSKGQPTLVVMELMAHGDLKSYLRSLRPEAENNPGRPPPTLQEMIQMAAEIADGMAYLNAKKFVHRDLAARNCMVAHDFTVKIGDFGMTRDIYETDYYRKGGKGLLPVRWMAPESLKDGVFTTSSDMWSFGVVLWEITSLAEQPYQGLSNEQVLKFVMDGGYLDQPDNCPERVTDLMRMCWQFNPKMRPTFLEIVNLLKDDLHPSFPEVSFFHSEENKAPESEELEMEFEDMENVPLDRSSHCQREEAGGRDGGSSLGFKRSYEEHIPYTHMNGGKKNGRILTLPRSNPS

SEQ ID NO: 48 - protein, 118 aa, synthetic construct
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVSVSS

SEQ ID NO: 49 - protein, 109 aa, synthetic construct
EIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVESKR

SEQ ID NO: 50 - protein, 107 aa, synthetic construct [sequence omitted]

SEQ ID NO: 51 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDQLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHNHYTQKSLSLSPGK

SEQ ID NO: 54 - protein, 764 aa, synthetic construct
MRPADLLQLVLLLDLPRDLGGMGCSSPPCECHQEEDFRVTCKDIQRIPSLPPSTQTLKLIETHLRTIPSHAFSNLPNISRIYVSIDVTLQQLESHSFYNLSKVTHIEIRNTRNLTYIDPDALKELPLLKFLGIFNTGLKMFPDLTKVYSTDIFFILEITDNPYMTSIPVNAFQGLCNETLTLKLYNNGFTSVQGYAFNGTKLDAVYLNKNKYLTVIDKDAFGGVYSGPSLLDVSQTSVTALPSKGLEHLKELIARNTWTLKKLPLSLSFLHLTRADLSYPSHCCAFKNQKKIRGILESLMCNESSMQSLRQRKSVNALNSPLHQEYEENLGDSIVGYKEKSKFQDTHNNAHYYVFFEEQEDEIIGFGQELKNPQEETLQAFDSHYDYTICGDSEDMVCTPKSDEFNPCEDIMGYKFLRIVVWFVSLLALLGNVFVLLILLTSHYKLNVPRFLMCNLAFADFCMGMYLLLIASVDLYTHSEYYNHAIDWQTGPGCNTAGFFTVFASELSVYTLTVITLERWYAITFAMRLDRKIRLRHACAIMVGGWVCCFLLALLPLVGISSYAKVSICLPMDTETPLALAYIVFVLTLNIVAFVIVCCCYVKIYITVRNPQYNPGDKDTKIAKRMAVLIFTDFICMAPISFYALSAILNKPLITVSNSKILLVLFYPLNSCANPFLYAIFTKAFQRDVFILLSKFGICKRQAQAYRGQRVPPKNSTDIQVQKVTHDMRQGLHNMEDVYELIENSHLTPKKQGQISEEYMQTVL